(19) United States
(12) Patent Application Publication
(10) Pub. No.: US 2024/0268721 A1
(43) Pub. Date: Aug. 15, 2024

(54) DISEASE MANAGEMENT SYSTEM

(71) Applicant: Medtronic, Inc., Minneapolis, MN (US)

(72) Inventors: Daniel Hahn, Orange, CA (US); Akhil Srinivasan, Woodland Hills, CA (US); Patrick W. Kinzie, Glendale, AZ (US); Randal C. Schulhauser, Phoenix, AZ (US); Jennifer L. Marckmann, Scottsdale, AZ (US); Mohsen Askarinya, Chandler, AZ (US); James K. Carney, Roseville, MN (US); David L. Probst, Chandler, AZ (US); Santhisagar Vaddiraju, Plymouth, MN (US); Alejo Chavez Gaxiola, Costa Mesa, CA (US); Richard J. O'Brien, Hugo, MN (US); Anna M. Tycon, Scottsdale, AZ (US); Omid Mahdavi, Fountain Hills, AZ (US); Shawn C. Kelley, Shoreview, MN (US); David A. Anderson, Stanchfield, MN (US)

(21) Appl. No.: 18/626,074

(22) Filed: Apr. 3, 2024

Related U.S. Application Data
(63) Continuation-in-part of application No. 18/460,271, filed on Sep. 1, 2023.
Application No. 18/460,271 is a continuation of application No. 16/116,346, filed on Aug. 29, 2018, now Pat. No. 11,744,492.

(51) Int. Cl. A61B 5/1468; A61B 5/053; A61B 5/145; A61B 5/1486; G01N 27/30; G01N 27/327

(52) U.S. Cl. CPC A61B 5/1468; A61B 5/053; A61B 5/14532; A61B 5/14546; A61B 5/1486; G01N 27/301; G01N 27/3271

(57) ABSTRACT

Techniques are disclosed for measuring an analyte in a biological system. A system may include a medical device with an electrochemical sensor configured to sense the concentration of a plurality of analytes present in a biological system. Processing circuitry of the system may retrieve, identify, and process a respective signal from a respective work electrode to determine the concentration of a respective analyte. The system may further include an implantable medical device configured to sense a cardiac electrogram (EGM). In some examples, the system may be configured to determine one or more patient-specific relationships between the respective signals of the electrochemical sensor and the cardiac EGM during a first period of time. Based on the patient-specific relationships, the system may estimate concentrations of the one or more analytes corresponding to the respective signals based on the cardiac EGM of the patient collected over a second period of time.

This application is a continuation in part of U.S. patent application Ser. No. 18/460,271, filed Sep. 1, 2023, which is a continuation of U.S. patent application Ser. No. 16/116,346, filed Aug. 29, 2018, now U.S. patent Ser. No. 11/744,492, the entire contents of each of which are incorporated herein by reference.

### FIELD

The present technology is related generally to methods and devices for measuring an analyte present in a biological system.

### BACKGROUND

Laboratory tests are often used to measure analyte concentrations in fluids, such as fluids in a biological system. For example, a basic metabolic panel (BMP) is a typical lab test that includes three types of serum markers measuring seven analyte concentrations: an electrolyte panel that includes measurement of the concentrations of sodium, chloride, potassium, and bicarbonate/carbon dioxide; a renal function test that includes measurement of the concentration of blood urea nitrogen (“BUN”) and creatinine; and a blood glucose test that includes measurement of the concentration of glucose. Other laboratory tests may be used to measure different analytes. A typical BMP, or other lab laboratory test, requires a biological sample, e.g., blood, be taken from a patient and analyzed by bench top and/or clinical equipment to determine analyte concentrations.

### SUMMARY

A system may include a medical device with an electrochemical sensor configured to sense the concentration of a plurality of analytes present in a biological system. The medical device may be insertable into the biological system, such as insertable transcutaneously into the interstitial fluid of a human patient. Each respective work electrode of a plurality of respective work electrodes of the electrochemical sensor may produce a respective signal indicative of a concentration of a respective analyte in the biological system. Processing circuitry of the system may retrieve, identify, and process a respective signal from a respective work electrode to determine the concentration of a respective analyte. In this way, the medical device may enable continuous or near continuous monitoring of the multiple analyte concentrations in a biological system.

The system may further include an implantable medical device configured to sense a cardiac electrogram (EGM), e.g., an electrocardiogram (ECG), of the patient. In some examples, the system may be configured to determine one or more patient-specific relationships between the respective signals of the electrochemical sensor and the cardiac EGM during a first period of time, e.g., an initialization period. Based on the patient-specific relationships, the system may estimate concentrations of the one or more analytes corresponding to the respective signals based on the cardiac EGM of the patient collected over a second period of time, e.g., a monitoring period.

In an example, the disclosure describes a system comprising: an electrochemical sensor comprising one or more work electrodes configured to produce one or more signals indicative of a concentration of one or more analytes in a patient; an implantable medical device (IMD) configured to sense a cardiac electrogram (EGM) of the patient; and processing circuitry in communication with the electrochemical sensor and the IMD, wherein the processing circuitry is configured to: receive from the electrochemical sensor the one or more signals indicative of the concentration of the one or more analytes over a first period of time; receive from the implantable medical device the cardiac EGM collected over the first period of time; determine one or more patient-specific relationships between the one or more signals and the cardiac EGM for the patient, based on the received one or more signals and the collected cardiac EGM over the first period of time; and based on the patient-specific relationships, determine an estimated concentration of the one or more analytes based on the EGM for the patient collected over a second period of time.

In another example, the disclosure describes a method comprising: producing, by an electrochemical sensor comprising one or more work electrodes, one or more signals indicative of a concentration of one or more analytes in a patient; sensing, by an implantable medical device (IMD), a cardiac electrogram (EGM) of the patient; receiving from the electrochemical sensor, by processing circuitry in communication with the electrochemical sensor and the IMD, one or more signals indicative of the concentration of the one or more analytes over a first period of time; receiving from the IMD, by the processing circuitry, the cardiac EGM collected over the first period of time; determining, by the processing circuitry, one or more patient-specific relationships between the one or more signals and the cardiac EGM for the patient, based on the received one or more signals and the collected cardiac EGM over the first period of time; and based on the patient-specific relationships, determining, by the processing circuitry, an estimated concentration of the one or more analytes based on the EGM for the patient collected over a second period of time.

In another example, the disclosure describes a non-transitory computer-readable storage medium, comprising instructions that, when executed by processing circuitry, cause the processing circuitry to: receive from an electrochemical sensor, one or more signals indicative of a concentration of a one or more analytes over a first period of time; receive from an implantable medical device a cardiac electrogram (EGM) collected over the first period of time; determine one or more patient-specific relationships between the one or more signals and the cardiac EGM for a patient, based on the received one or more signals and the collected cardiac EGM over the first period of time; and based on the patient-specific relationships, determine an estimated concentration of the one or more analytes based on the EGM for the patient collected over a second period of time.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 illustrates an example environment of a medical device system in conjunction with a patient, in accordance with one or more techniques of this disclosure.

FIG. 2A is a schematic and conceptual diagram illustrating a cross-sectional side view of an example electrochemical sensor including a plurality of dielectric barriers between each pair of adjacent electrodes of a plurality of electrodes, in accordance with one or more techniques of this disclosure.

FIG. 2B is a schematic and conceptual diagram illustrating a cross-sectional side view of an example plurality of respective work electrodes with each respective work electrode of the plurality of respective work electrodes having a selected chemistry, in accordance with one or more techniques of this disclosure.

FIG. 3 is a schematic and conceptual partial circuit diagram illustrating an example medical device that includes an electrochemical sensor including a counter electrode, a common reference electrode, and a work platform having a plurality of respective work electrodes operatively coupled to corresponding electrical components, in accordance with one or more techniques of this disclosure.

FIG. 4A is a schematic and conceptual block diagram illustrating an example medical device configured to be inserted into the interstitial fluid of a patient, in accordance with one or more techniques of this disclosure.

FIG. 4B is a schematic and conceptual block diagram illustrating example processing circuitry of the example medical device of FIG. 4A, in accordance with one or more techniques of this disclosure.

FIG. 5 is a functional block diagram illustrating an example configuration of the IMD of FIG. 1, in accordance with one or more techniques described herein.

FIG. 6 is a block diagram illustrating an example configuration of components of the external device of FIG. 1, in accordance with one or more techniques of this disclosure.

FIG. 7 is a flow chart illustrating an example operation for determining an analyte concentration, in accordance with one or more techniques of this disclosure.

FIG. 8 is a flow chart illustrating an example operation for determining an estimated concentration of an analyte based on cardiac electrogram (EGM) data, in accordance with one or more techniques of this disclosure.

FIG. 9 is a flow chart illustrating an example operation for calibrating a new sensor portion based on cardiac EGM data, in accordance with one or more techniques of this disclosure.

The details of one or more examples of this disclosure are set forth in the accompanying drawings and the description below. Other features, objects, and advantages of this disclosure will be apparent from the description and drawings, and from the claims.

### DETAILED DESCRIPTION

A system may include a medical device comprising an electrochemical sensor, processing circuitry, an antenna, and a power source. The electrochemical sensor may include a common reference electrode, at least one counter electrode, and a work electrode platform having a plurality of respective work electrodes. In some examples, the common reference electrode and the at least one counter electrode may be operatively coupled to each work electrode of the plurality of respective work electrodes. Using a single, common reference electrode and, in some examples, a single counter electrode may reduce the size of the electrochemical sensor.

Each respective work electrode of the plurality of respective work electrodes may include a respective reagent substrate configured to react with a respective analyte, e.g., an analyte present in a sample fluid to which the plurality of respective work electrodes are exposed. In some examples, a respective membrane disposed on the respective reagent substrate, such as a limiting membrane and/or a selective ion transfer membrane, may be selectively permeable to the respective analyte and used to control the extent or rate of reaction of the analyte at a surface of the reagent substrate, e.g., by controlling a rate of exposure of the reagent substrate to the analyte. In this way, the chemistry of the respective work electrode may be selected to be specific to a respective analyte. In some examples, a reaction of the respective analyte with the respective reagent substrate, e.g., an oxidation reaction or a reduction reaction, may produce, or at least partially cause the generation of, a respective signal indicative of a respective concentration of the respective analyte. In some examples, an interaction of the respective analyte with the respective reagent substrate, e.g., at the double layer, may produce, or at least partially cause the generation of, a respective signal indicative of a respective concentration of the respective analyte. In some examples, the respective signal may include an electrical signal resulting from a change in current, potential, or impedance at the respective work electrode. In this way, the plurality of respective work electrodes may produce respective signals indicative of respective analytes.

In some examples, the system may additionally include an implantable medical device (IMD). In some examples, the IMD may be configured to sense a cardiac electrogram (EGM) of a patient, such as an electrocardiogram (ECG) of the patient. Based on the cardiac EGM, the system may estimate one or more analyte concentrations. In this way, the system may determine the one or more analyte concentrations based on one or both of the electrochemical sensor signals and the EGM. In this way, the system may determine the one or more analyte concentrations with relatively higher accuracy, e.g., relative to the electrochemical sensor or EGM alone.

Processing circuitry of the system may additionally be configured to identify patient-specific relationships between the one or more signals from the electrochemical sensor and the cardiac EGM of the IMD collected over a first time period. Upon identifying patient-specific relationships between the one or more signals and the cardiac EGM, the system may be able to determine a patient-specific, estimated concentration of the one or more analytes based on the cardiac EGM of the patient over a second period of time. The techniques of this disclosure may advantageously allow the system to determine one or more patient-specific estimations of analyte concentrations based on the cardiac EGM, which may allow the patient or a clinician to monitor patient analyte concentrations without the patient wearing or otherwise using an electrochemical sensor for a period of time, e.g., weeks, months, or years, which may improve patient quality of life, by, for example, decreasing patient burden of continuously wearing or otherwise using the electrochemical sensor.

In some examples, the electrochemical sensor may include a replaceable portion, e.g., a replaceable work electrode platform. In some examples, the clinician or patient may want to calibrate a replacement portion, i.e., a new sensor portion, and/or ensure the new portion is outputting accurate analyte concentrations. By determining patient-specific relationships between the electrochemical sensor signals from a first work electrode platform and the EGM during the first time period, the techniques of this disclosure may advantageously facilitate calibration of the new sensor portion based on a cardiac EGM of the patient after the new sensor portion has been installed. By facilitating calibration of the new sensor portion, the techniques of this disclosure may decrease clinician burden, e.g., by decreasing review time of new sensor portions. The techniques of this disclosure may additionally improve patient outcomes by ensuring accurate analyte concentration estimations.

In some examples, a patient may suffer one or more disease states, e.g., diabetes, heart failure, and/or kidney failure. In some examples, the one or more disease states may be associated with one or more comorbidities. For example, patients with diabetes may have an increased risk of developing heart failure and/or kidney failure. A medical device system may monitor a patient disease state, e.g., diabetes. In the example of diabetes, the one or more analyte concentrations may include a glucose concentration and a potassium concentration, which may be used to monitor and facilitate therapy administration of the diabetic patient. In some examples, the glucose concentration and the potassium concentration may additionally be indicative of changes in other patient disease state statuses. For example, changes in patient glucose concentrations and potassium concentrations allow the system to estimate a heart rate variability of the patient. Changes in heart rate variability may be indicative of an onset of heart disease and/or kidney disease. In some examples, the system may implement a machine learning model to determine the patient heart rate variability and/or to identify a change in disease state, e.g., a change in risk of comorbidity onset. By monitoring the one or more analytes, e.g., glucose and potassium, the techniques of this disclosure may therefore facilitate identification of the onset of comorbidities, which may improve patient outcomes.

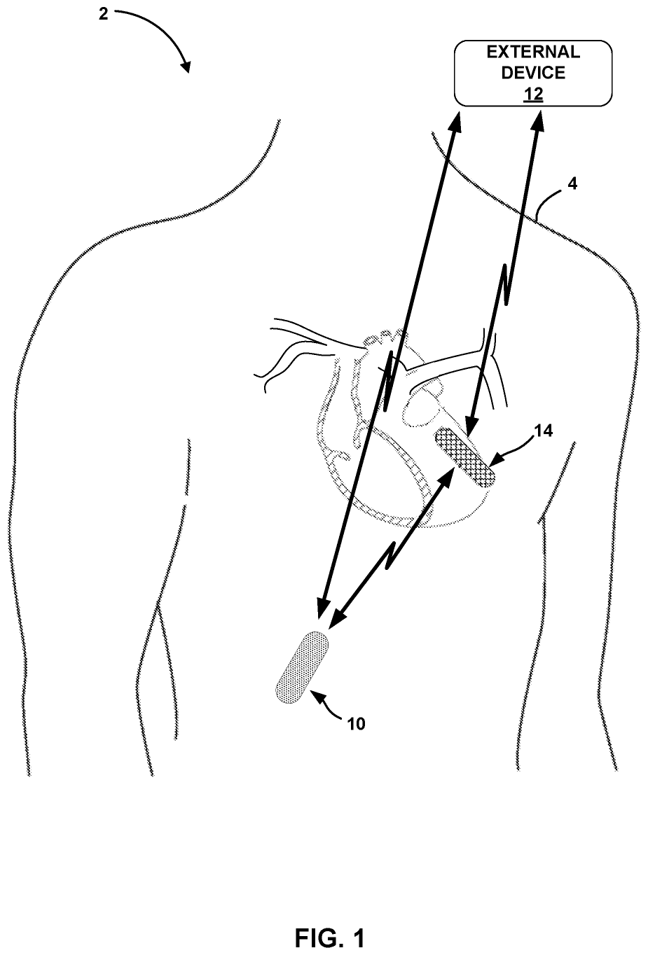

FIG. 1 illustrates an example environment of a medical device system 2 in conjunction with a patient 4, in accordance with one or more techniques of this disclosure. The example techniques may be used with one or more patient sensing devices, e.g., including an electrochemical sensor 10 and an implantable medical device 14, which may be in wireless communication with one or more computing devices (e.g., external device 12) and/or one another. As an example, electrochemical sensor 10 is positioned on an abdomen of patient 4. However, electrochemical sensor 10 may be positioned on a different location of patient 4, such as an arm of patient 4. Although not illustrated in FIG. 1, electrochemical sensor 10 may include one or more work electrodes to sense signals corresponding to one or more analyte concentrations, e.g., potassium concentration, glucose concentration, creatinine concentration, sodium concentration, chloride concentration, bicarbonate/carbon dioxide concentration, and/or blood urea nitrogen concentration, of patient 4.

In some examples, a user may monitor a patient disease state (e.g., diabetes, cardiorenal metabolic disease, cardiorenal syndrome). In examples in which the user is monitoring a specific patient disease state, it may be advantageous, e.g., to reduce the size of electrochemical sensor 10, to monitor a particular subset of the potential analyte concentrations, such as, in the example of a diabetic patient, the potassium concentration and the glucose concentration. For example, potassium concentration and glucose concentration may be important indicators of patient health and treatment efficacy of the diabetic patient. Additionally, potassium concentration and glucose concentration may be indicative of patient comorbidities, such as heart failure and/or kidney failure.

As an example, IMD 14 is depicted as being implanted outside a thoracic cavity of patient 4 (e.g., subcutaneously in the pectoral location illustrated in FIG. 1). However, IMD 14 may be implanted elsewhere in the patient. IMD 14 may be positioned near the sternum near or just below the level of the heart of patient 4, e.g., at least partially within the cardiac silhouette, and be configured to sense an ECG and/or other physiological signals from that position. In some examples, IMD 14 takes the form of the Reveal LINQ™ or LINQ II™ insertable cardiac monitor (ICM), available from Medtronic, Inc., of Minneapolis, Minnesota.

Although described primarily in the context of examples in which IMD 14 takes the form of an ICM, the techniques of this disclosure may be implemented in systems including any one or more implantable or external medical devices, including monitors, pacemakers, defibrillators (e.g., subcutaneous or substernal), wearable external defibrillators (WAEDs), neurostimulators, drug pumps, patch monitors, or wearable physiological monitors, e.g., wrist or head wearable devices. Examples with multiple IMDs or other sensing devices may be able to collect different data useable by system 2. Although not illustrated in FIG. 1, IMD 14 includes electrodes and/or other sensors to sense a cardiac EGM, e.g., an ECG, of patient 4 and may collect and store ECG data based on the sensed ECG signal.

In some examples, electrochemical sensor 10 and IMD 14 are configured to be in wireless communication with each other. In some examples, IMD 14 may transmit data to electrochemical sensor 10. Additionally, or alternatively, electrochemical sensor 10 may transmit data to IMD 14. Electrochemical sensor 10, IMD 14, or a combination thereof may transmit data, e.g., signal data indicative of analyte concentrations and/or cardiac EGM data, to external device 12.

External device 12 may be a computing device with a display viewable by the user and an interface for providing input to external device 12 (i.e., a user input mechanism). External device 12 may be configured for wireless communication with IMD 14 and electrochemical sensor 10. In some examples, external device 12 retrieves sensed cardiac EGM data from IMD 14 and one or more signals indicative of one or more analyte concentrations from electrochemical sensor 10 that were collected and stored by IMD 14 and electrochemical sensor 10, respectively. In some examples, external device 12 takes the form of a personal computing device of patient 4. For example, external device 12 may take the form of a smartphone of patient 4. In some examples, external device 12 may be any computing device configured for wireless communication with IMD 14, such as a desktop, laptop, or tablet computer. External device 12 may communicate with IMD 14 and electrochemical sensor 10 via near-field communication technologies e.g., inductive coupling, NFC or other communication technologies operable at ranges less than 10-20 cm, and far-field communication technologies, e.g., radiofrequency telemetry according to the Bluetooth® or Bluetooth® Low Energy (BLE) protocols, or other communication technologies operable at ranges greater than near-field communication technologies. When external device 12 is configured for use by the clinician, external device 12 may be used to transmit instructions to IMD 14 and/or electrochemical sensor 10. The clinician may also configure and store operational parameters for IMD 14 and/or electrochemical sensor 10 with the aid of external device 12. In some examples, external device 12 assists the clinician in the configuration of IMD 14 and electrochemical sensor 10 by providing a system for identifying potentially beneficial operational parameter values.

External device 12 may be used to retrieve data from IMD 14 and/or electrochemical sensor 10. The retrieved data may include cardiac EGM data, e.g., ECG data, measured by IMD 14 based on ECG signals sensed by IMD 14 and analyte concentration data measured by electrochemical sensor 10 based on signals indicative of analyte concentrations sensed by electrochemical sensor 10. External device 12 may retrieve data continuously or on a regular transmission schedule, e.g., hourly, daily, weekly, etc. In some examples, external device 12 may retrieve data when prompted to by a user.

Processing circuitry of system 2, e.g., of IMD 14, electrochemical sensor 10, external device 12, and/or one or more other computing devices (not illustrated in FIG. 1) may be configured to perform the example techniques described herein, e.g., the techniques for determining analyte concentrations based on cardiac EGM data collected by IMD 14 and/or signal data indicative of analyte concentrations collected by electrochemical sensor 10.

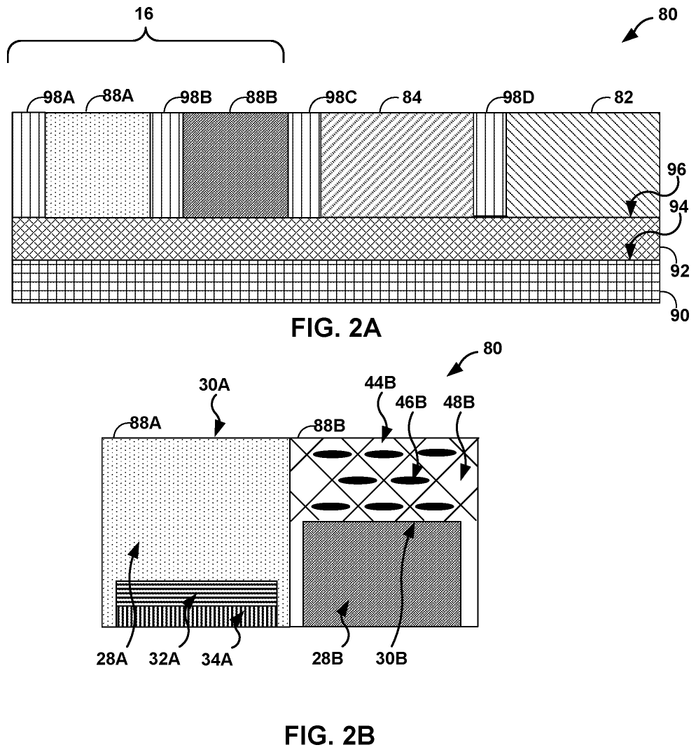

FIG. 2A is a schematic and conceptual diagram illustrating a cross-sectional side view of an example electrochemical sensor 10 including at least one counter electrode 82, a common reference electrode 84, and a work platform 16 including a plurality of respective work electrodes 88A and 88B (collectively, “work electrodes 88”) and a plurality of dielectric barriers 98A, 98B, 98C, and 98D between each electrode, in accordance with one or more techniques of this disclosure. In some examples, electrochemical sensor 10 includes fewer or more electrodes. For example, electrochemical sensor 10 may include only counter electrode 82 and work platform 16, only common reference electrode 84 and work platform 16, more than one counter electrode 82, more than one reference electrode 84, or more than two work electrodes 88, such as five work electrodes 88, seven work electrodes 88, ten work electrodes 88, or more.

In the example illustrated in FIG. 2A, electrochemical sensor 10 includes a dielectric substrate layer 90 defining a first major surface 94. In some examples, dielectric substrate layer 90 may include a biocompatible polymer, such as polyamide or polyimide, liquid crystal polymer, silica glass, such as a glass wafer, sapphire, such as a sapphire wafer, or silicon. In some examples, first major surface 94 is substantially planar. In other examples, first major surface 94 may include surface features, such as ridges, valleys, or apertures, corresponding to features such as electrical traces or through vias. Surface features on or in first major surface 94 may be formed by any suitable means, such as, for example, machining, laser etching, chemical etching, or semiconductor manufacturing techniques such as front-end-of-line (FEOL) processes. In this way, dielectric substrate layer 90 may be formed to support additional layers, facilitate manufacture of the electrochemical sensor 10, or both.

An interconnect layer 92 is on first major surface 94 of dielectric layer 90. Interconnect layer 92 includes an electrically conductive material, such as, for example, aluminum, cadmium, chromium, copper, gold, nickel, platinum, titanium, indium nitride, indium phosphide, zinc oxide, alloys thereof, or the like. In some examples, first major surface 94 may be metallized by, for example, chemical vapor deposition, physical vapor deposition, thermal spraying, cold spraying, or the like, to form interconnect layer 92. Interconnect layer 92 defines a second major surface 96 opposite first major surface 94. Counter electrode 82, common reference electrode 84, and work platform 16 may be disposed on second major surface 26 to electrically couple each respective work electrode of work electrodes 18 to one or both of counter electrode 82 and common counter electrode 84. In some examples, interconnect layer 22 may be operatively coupled to a computing device, such as processing circuitry, to facilitate transmission of a signal from a respective work electrode of work electrodes 88 to the computing device. In some examples, interconnect layer 92 may form a plurality of electrical traces, e.g., formed using semiconductor manufacturing techniques such as back-end-of-line (BEOL) processes. A respective electrical trace or the plurality of electrical traces may electrically couple a respective work electrode of work electrodes 88 to one or more of a computing device, counter electrode 82, or common reference electrode 84.

Electrochemical sensor 10 is configured to detect the concentration of each of a plurality of analytes present in a sample fluid. In some examples, the sample fluid may include a biological fluid, such as blood, interstitial fluid, saliva, urine, spinal fluid, peritoneal fluid, or the like. In some examples, the plurality of analytes include, but are not limited to, one or more of sodium, chloride, potassium, bicarbonate/carbon dioxide, blood urea nitrogen (“BUN”), creatinine, glucose, brain natriuretic peptide (BNP), C-reactive protein (CRP), troponin I (cTnI), lactate, pH, L-dopa, and the like. Each respective work electrode of work electrodes 88 and, in some examples, counter electrode 82 and/or common reference electrode 84, may be fluidly coupled to the sample fluid. In this way, electrochemical sensor 10 may enable continuous or near continuous monitoring of the multiple analyte concentrations in the sample fluid. By using a common reference electrode and, optionally, one or more counter electrodes that are shared among two or more respective work electrodes, a size of electrochemical sensor 10 may be reduced. The configuration of work platform 16, counter electrode 82, and common reference electrode 84 of FIGS. 2A and 2B is merely provided as an example. A plurality of configurations is possible. Counter electrode 82, common reference electrode 84, and work platform 16 may be oriented differently, e.g., to reduce the surface area of electrochemical sensor 10. For example, work electrode platform 16 may be stacked on counter electrode 82 and common reference electrode 84.

Counter electrode 82 (e.g., auxiliary cell) may be disposed on interconnect layer 92. Counter electrode 82 may be configured to function as a cathode when a respective work electrode of work electrodes 88 is operating as an anode or vice versa. In some examples, counter electrode 82 may include an electrochemically inert material, such as copper, gold, indium tin oxide, platinum, silver, silver/silver chloride, titanium, tungsten, tantalum, alloys thereof, carbon, or conductive nanoparticles embedded within a polymeric material. Counter electrode 82 may include any suitable shape, such as rectilinear or curvilinear. In some examples, counter electrode 82 may define a rectangular shape. In some examples, a length of counter electrode 82 is between approximately 0.2 millimeters and approximately 1 centimeter, such as approximately 8.5 millimeters. In some examples, a width of counter electrode 82 is between approximately 0.2 millimeters and approximately 1 centimeter, such as approximately 8.5 millimeters. In some examples, counter electrode 82 may include a surface area larger than each respective work electrode of work electrodes 88. For example, counter electrode 82 may include a surface area that is approximately two to one hundred times the surface area of each respective work electrode of work electrodes 88. In some examples, the larger surface area of counter electrode 82 relative to work electrodes 88 may ensure that a half-reaction occurring at counter electrode 82 may occur fast enough so as not to limit the reactions at work electrodes 88.

In some examples, counter electrode 82 and a respective work electrode of work electrodes 88 may be configured to form a circuit over which current is either applied or measured. The potential of counter electrode 82 may be adjusted to balance a respective reaction occurring at a respective work electrode of work electrodes 88. In this way, the potential of the respective work electrode of work electrodes 88 may be measured against common reference electrode 84 without passing current over common reference electrode 4, which may compromise the stability of common reference electrode 84. In some examples, counter electrode 82 may be separated from work electrodes 88 by, for example, a dielectric barrier and/or orientation of work electrodes 18 with respect to counter electrode 82, to reduce byproducts generated at counter electrode 82 from contaminating the sample fluid. For example, if a reduction reaction is being performed at a respective work electrode of work electrodes 88, oxygen may be evolved from counter electrode 82.

Electrochemical sensor 10 includes dielectric barriers 98A, 98B, 98C and 98D (collectively, “dielectric barriers 98”) between each pair of adjacent electrodes, e.g., work electrodes 88, counter electrode 82 and common reference electrode 84.

In some examples, dielectric barriers 98 may be configured to reduce electrical interference between adjacent electrodes. For example, dielectric barriers 98 may reduce electron transfer between adjacent electrodes, reduce electromagnetic interference between adjacent electrode, or both. In some examples, dielectric barriers 98 may include a biocompatible polymer, such as polyamide or polyimide, liquid crystal polymer, silica glass, such as a glass wafer, sapphire, such as a sapphire wafer, or silicon. In some examples, dielectric barriers 98 may be integrally formed with dielectric substrate 90. For example, each respective work electrode of work electrodes 88, counter electrode 82, and common reference electrode 84 may be disposed within a cavity defined by dielectric substrate 90.

Common reference electrode 84 may be configured to provide a stable and known electrode potential. In some examples, common reference electrode 84 may provide a stable potential by using a redox based system. For example, common reference electrode 84 may include a silver/silver chloride electrode having a potential of about 0.197 volts. Common reference electrode 84 including other materials may have a different stable and known electrode potential. In some examples, common reference electrode 84 may include gold, platinum, silver/silver chloride, hydrogen electrode, copper sulfate, or palladium. Common reference electrode 84 may include any suitable shape, such as rectilinear or curvilinear. In some examples, common reference electrode 84 may define a rectangular shape. In some examples, a length of common reference electrode 84 is between approximately 0.2 millimeters and approximately 1 centimeter, such as approximately 8.5 millimeters. In some examples, a width of common reference electrode 84 is between approximately 0.2 millimeters and approximately 1 centimeter, such as approximately 8.5 millimeters. In some examples, electrochemical sensor 10 may use an external driving voltage. In examples in which a driving voltage is applied to a respective work electrode of work electrodes 88, common reference electrode 84 may stabilize the driving voltage at the respective work electrode of work electrodes 88.

Each respective work electrode of work electrodes 88 may include a selected chemistry. For example, each respective work electrode of work electrodes 88 includes a respective reagent substrate disposed on second major surface 96. In some examples, a reaction of a respective analyte with a corresponding respective reagent substrate may cause electron transfer between a respective work electrode of work electrodes 88 and interconnect layer 92 (e.g., producing a current). In some examples, a reaction of a respective analyte with a corresponding respective reagent substrate may contribute to the potential in a respective work electrode of work electrodes 88 (e.g., producing a voltage). In some examples, interaction of a respective analyte with a corresponding respective reagent substrate may contribute to the resistivity of a respective work electrode of work electrodes 88 (e.g., changing an impedance of the respective work electrode of work electrodes 88 at the double layer). In this way, electrochemical sensor 10 may produce a current, a potential, or an impedance that may be processed by, for example, processing circuitry operatively coupled to each respective work electrode of work electrodes 88, and which allows detection of an analyte.

Each respective work electrode of work electrodes 88 may include any suitable shape, such as rectilinear or curvilinear. In some examples, each work electrode of work electrodes 88 may define a rectangular shape. In some examples, a length of each respective work electrode of work electrodes 88 is between approximately 0.1 millimeters and approximately 2.5 millimeters, such as approximately 0.5 millimeters. In some examples, a width of each respective work electrode of work electrodes 88 is between approximately 0.1 millimeters and approximately 2.5 millimeter, such as approximately 0.5 millimeters.

Each respective work electrode of work electrodes 88 may include one or more layers of materials to enable the respective work electrode of work electrodes 88 to produce a signal in response to the presence of a respective selected analyte. FIG. 2B is a schematic and conceptual diagram illustrating a cross-sectional side view of an example plurality of respective work electrodes 88 with each respective work electrode of the plurality of respective work electrodes 88 having a selected chemistry. As illustrated in FIG. 2B, each respective work electrode of work electrodes 88 may include a respective reagent substrate 28A and 28B (collectively, “reagent substrates 28”) configured to react with a respective analyte or a derivative thereof. For example, work electrode 88A may include reagent substrate 28A. In some examples, a respective analyte may interact with a surface 30A of a respective reagent substrate 28A. For example, the respective analyte may transfer electrons to surface 30A or remove electrons from surface 30A. In some examples, a respective work electrode of work electrodes 88 may include one or more conductive material layers. For example, work electrode 88A may include a first conductive layer 32A and a second conductive layer 34A. Example conductive material layers include, but are not limited to, gold, indium tin oxide, carbon, carbon paste, mesoporous carbon, carbon walled, platinum, shiny platinum, black platinum, polyimide silver, and silver/silver-chloride. In some examples, first conductive layer 32A may include a silver/silver-chloride material. In some examples, second conductive layer 34A may define a surface on which first conductive layer 32A may be disposed. First and second conductive material layers 32A and 34A may facilitate the transfer of electrons to or from interconnect layer 22.

In some examples, a respective reagent substrate of reagent substrates 28 includes a respective immobilization substrate configured to immobilize a respective reagent. In some examples, a respective reagent may include at least one enzyme, such as an oxidase enzyme. In some examples, a respective reagent may be immobilized on an immobilization substrate by, for example, physical entrapment (e.g., a respective reagent physically unable to pass through pores of the immobilization substrate), chemical bonding (e.g., ionic bonding, covalent bonding, van der Waals forces, and the like), or combinations thereof. In some examples, the immobilization substrate may include a polymer, such as polylysine, aminosilane, epoxysilane, or nitrocellulose, or a substrate having a three-dimensional lattice structure, such as a hydrogel, an organogel, or a xerogel. In some examples, the immobilization substrate may include a ligand configured to chemically bond to at least a portion of a respective reagent. For example, a respective immobilization substrate including glutaraldehyde may immobilize glucose oxidase. A respective immobilization substrate including primary amine conjugation enniatin may immobilize (used for sodium Na+ detection) can be immobilized to the working electrode through. In some examples, the immobilization substrate may include, but is not limited to, glutaraldehyde, thiol based conjugation compounds (e.g., 16-mercaptohexadecanoic acid (MHDA), diethyldithiocarbamic acid (DSH), dithiobissuccinimidylundecanoate (DSU), purine conjugation compounds, streptavidin-biotin conjugation compounds, a primary amine and a vinyl pyridine polymer, lysine, 1-ethyl-3-(3-dimethylaminopropyl)carbodiimide hydrochloride (EDC) and N-hydroxysuccinimide (NHS) coupling, agarose based gel and polymer mixtures, silane crosslinker, (hydroxyethyl)methacrylate, and poly(ethylene glycol) diacrylate polymer. By immobilizing a respective reagent, the immobilization substrate may reduce loss of the respective reagent to the sample fluid.

In examples in which a respective reagent substrate of reagent substrates 28 includes at least one enzyme, the at least one enzyme may be selected based on the analyte to be detected with the respective work electrode of work electrodes 88. For example, the at least one enzyme may be selected from the group consisting of glucose oxidase (for detecting glucose), creatinine amidohydrolase (for detecting creatinine), creatine amidinohydrolase (for detecting creatine), sarcosine oxidase (for detecting sarcosine), carbonic anhydrase (for detecting bicarbonate and/or carbon dioxide), choline oxidase (for detecting choline), horseradish peroxidase (for detecting peroxide, oxygen, nitric oxide, biogenic amines, or the like), thiamine oxidase (for detecting thiamine), urease (for detecting urea), glycerol-3-phosphate oxidase (for detecting glycerol-3-phosphate), L-amino acid oxidase (for detecting L-amino acid, such as, e.g., L-alanine), lactate oxidase (for detecting lactate and/or lactic acid), catalase (for detecting hydrogen peroxide, e.g., produced by other enzymatic reactions), alkaline phosphatase (for detecting phosphate esters), alcohol oxidase (for detecting primary alcohols), D-amino acid oxidase (for detecting D-amino acids, such as, e.g., D-serine), cholesterol oxidase (for detecting cholesterol), pyridoxal oxidase (for detecting pyridoxal), NAD(P)H oxidase (for detecting NAD(P)H), and pyruvate oxidase (for detecting pyruvate), or mixtures thereof. In some examples, the at least one enzyme may be selected to react with a selected analyte and provide a reaction pathway to enable detection of the concentration of the selected analyte.

In examples in which a respective reagent substrate of reagent substrates 28 includes glucose oxidase (e.g., notatin), glucose oxidase may oxidize glucose in the sample fluid to produce D-glucono-δ-lactone and hydrogen peroxide. The liberated hydrogen peroxide may be oxidized at, e.g., second major surface 26 or first conductive material layer 32A, to produce an electric current that is proportional to the glucose concentration in the sample fluid.

In some examples, a selective ion transfer membrane may include an ionophore membrane. For example, as illustrated in FIG. 2B, work electrode 18B may include reagent substrate 28B defining surface 30B and ionophore membrane 44B. In some examples, ionophore membrane 44B may include a plurality of ionophores 46B dispersed in an ionophore matrix material 48D. Plurality of ionophores 46B may be selected to be preferentially permeable to a selected ion or group of ions. In some examples, plurality of ionophores 46B may include, but is not limited to, crown ethers, cryptands, calixarenesm, phenols, amino methylated polystyrene salicylaldehyde, beauvericin, calcimycine, cezomycin, carbonyl cyanide m-chlorophenyl hydrazone, dibenzo-18-crown-6, enniatin, gramicidin A, ionomycin, lasalocid, macrolides, monensin, nigericin, nigericin sodium salt, narasin, nonactin, polyimide/lycra blend, salinomycin, tetronasin, valinomycin, potassium ionophore III (BME 44) or mixtures thereof. Ionophore matrix material 48D may include, but it not limited to, polyvinylchloride, silicone, fluorosilicone, polyurethane, glutaraldehyde, UV curable polymers like PVA-SbQ, PVA hydrogels, pHEMA-HAA crosslinking, and agarose gel.

Various signal processing techniques may be used to detect analytes or concentrations of analytes. For example, one or more of amperometry, potentiometry, and/or electrochemical impedance spectroscopy (EIS) may be used to analyze signals from work electrodes 88. In some examples, a plurality of signal processing techniques may be used to detect a respective analyte or a respective concentration of a respective analyte. For example, two or more respective work electrodes of work electrodes 88 may be configured to detect a respective analyte, where each of the two or more respective work electrodes of work electrode 88 use different signal processing techniques.

One example signal processing technique may include amperometry. Amperometry may be used to measure the reduction or oxidation of a respective analyte at a respective work electrode of work electrodes 88. In examples using amperometry, a working potential applied between a respective work electrode of work electrodes 88 and common reference electrode 84 may generate a current that is carried between the respective work electrode of work electrode 88 and common reference electrode 84. The current may be measured using, for example, an ammeter, a current to frequency converter, or a current to voltage converter, such as a resistor in the current path or a transimpedance amplifier. The current may change as a respective analyte is oxidized or reduced at the respective work electrode of work electrodes 88 (e.g., as electrons are produced by an oxidation reaction or consumed a reduction reaction). For example, the current may be related to the rate of reaction (V_{A}) by the expression i=nFAV_{A}, wherein n is the number of electrons per mole (or the number of electrons per molecule), F is Faraday's constant, and A is the surface area of the respective work electrode. The number of electrons transferred to the respective work electrode, n, may be proportional to the concentration of the analyte in the sample fluid. In this way, the measured current may be associated with the concentration of the analyte in the sample fluid.

In some examples, the applied potential may be adjusted to maximize the response for the analyte of interest while minimizing the response for interfering analytes. For example, a respective analyte may have a higher affinity for a selected working potential or range of working potentials. In some examples, the working potential may be pulsed (e.g., a duration of about one hundred to about nine hundred milliseconds). The pulsed working potential may be followed by a higher potential or a lower potential to at least partially clean the respective analyte from the respective work electrode of work electrodes 88 (e.g., reduce the affinity of the respective analyte for the respective work electrode). In examples in which the working potential is pulsed, the current may be measured only while the working potential is applied.

Potentiometry may be used to measure the potential between two electrodes in a sample fluid. In examples using potentiometry, common reference electrode 84 may have a constant potential irrespective of the concentration of analytes in the sample fluid. A respective work electrode of work electrodes 88 may demonstrate Nernstian response to the composition of the sample fluid. That is, a difference of potential between common reference electrode 84 and the respective work electrode of work electrodes 88 may be proportional to the concentration of the analyte in the sample fluid, e.g., the difference of potential may increase approximately 59 mV for every order of magnitude increase in the concentration of the analyst in the sample fluid. In some examples, a respective work electrode of work electrodes 88 may include a selective ion transport membrane. For example, the selective ion transport membrane may include an ionophore to control transport of a respective analyte to the respective work electrode of work electrodes 88. In some examples, the ionophore may control the transport of, for example, hydrogen ions (H⁺), sodium ions (Na⁺), potassium ions (K⁺), chloride ions (Cl⁻), calcium ions (Ca²⁺), bicarbonate (HCO₃⁻), and/or BUN. In this way, a respective work electrode of work electrodes 88 may convert the activity of a respective analyte in the sample fluid into an electrical potential.

The electrical potential may be measured by, for example, a voltmeter, such as a high output impedance amplifier. The measured voltage may be proportional to the ionic activity of the respective analyte according to the Nernst equation. For example, the Nernst equation relates the reduction potential of an electrochemical reaction (half-cell or full cell reaction) to the standard electrode potential, temperature, and activities (often approximated by concentrations) of the chemical species undergoing reduction and oxidation. In one example, the Nernst equation may be given by E_{cell}=E°+2.3026(RT/zF)log₁₀(Qᵣ), where E_{cell} is the cell potential (electromotive force, emf) at the temperature of interest, E° is the standard cell potential (millivolts), R is the universal gas constant (Joules per kelvin-mole), T is the temperature (kelvin), z is the number of electrons transferred to the respective work electrode of work electrodes 88, F is Faraday's constant (coulombs per mole of electrons), and Qᵣ is the reaction quotient of the cell reaction. The number of electrons transferred to the respective work electrode, z, may be proportional to the concentration of the analyte in the sample fluid. In this way, the measured potential may be associated with the concentration of the analyte in the sample fluid.

Electrochemical impedance spectrometry (EIS) is a perturbative characterization of the dynamics of an electrochemical process by determining an impedance of a respective work electrode of work electrodes 88 in a sample fluid (e.g., the electrochemical system) in response to a potential applied to the respective work electrode of work electrodes 88. A current frequency dependence of the impedance of the respective work electrode of work electrodes 88 may be associated with the concentration of a respective analyte in the sample fluid. For example, the limiting membrane of a respective work electrode of work electrodes 88 may be selected to enable approximately steady state diffusion of a target analyte to and from the respective work electrode. A working potential may be applied to the respective work electrode of work electrodes 88, where the working potential may include a direct current polarization potential and a superimposed alternating current potential having a selected frequency (e.g., an excitation signal). The current response (e.g., response signal) may be measured by, for example, an ammeter. The selected frequency may include a frequency predetermined to result in a response signal for a selected analyte (e.g., an optimal frequency for the selected analyte). Additionally, or alternatively, the selected frequency may include a plurality of frequencies applied sequentially, such as, for example, ranging from 1 Hz to 100 kHz (e.g., a frequency sweep). In some examples, the working potential may be selected to the dynamic noise for EIS. In examples in which the excitation signal is sufficiently small, e.g., between approximately 1 millivolts (mV) to 10 mV, the current response may be modeled as a linear electrochemical system.

By using a common reference electrode 84 and, optionally, at least one counter electrode 82 that are shared among two or more respective work electrodes 88, a size of electrochemical sensor 10 may be reduced. Reducing the size of electrochemical sensor 10 may enable incorporating electrochemical sensor 10 into a medical device that may be inserted in a patient. Inserting the medical device into the patient may enable continuous or near continuous monitoring of the concentration of an analyte in interstitial fluid of the patient. In this way, the patient may remain ambulatory during monitoring of the concentration of an analyte in interstitial fluid of the patient. Additionally, the concentration of an analyte in interstitial fluid of the patient may be monitored with an increased frequency compared to other methods of monitoring the concentration of an analyte. Increasing the frequency of monitoring may improve the quality of analyte concentration data to improve patient care.

When modeled as a linear electrochemical system, the impedance with respect to radial frequency, Z(ω), may be represented as a complex number (based on Euler's relationship exp(j φ)=cos(φ)−jsin(φ)) as Z(ω)=Zₒ(cos(φ)−jsin(φ)), where Zₒ is associated with the working potential, and φ is the phase shift of the response signal. In some examples, the impedance Z(ω) may be used to produce a Nyquist plot (e.g., real part of the expression for Z(ω) plotted on the X-axis and the imaginary part of the expression for Z(ω) is plotted on the Y-axis). In some examples, the impedance Z(ω) may be used to produce a Bode Plot (e.g., log frequency on the X-axis and both the absolute values of the impedance (|Z|=Z₀) and the phase-shift on the Y-axis). In some examples, modulus, admittance, and capacitance may be used to represent the current response and/or transformations thereof. In examples in which the electrochemical process is dependent on diffusion of the respective analyte, the impedance may have a low-frequency character, which may be modeled as a Warburg impedance element. In some examples, an equivalent circuit model, e.g., a Randles circuit model, may be used to process the measured current response to determine the impedance the electrochemical system. In some examples, the double layer of the electrochemical system may be modeled as an imperfect parallel plate capacitor (or a constant phase element), such that the concentration of the analyte may be associated with the determined impedance. In this way, the EIS may be used to determine a concentration of the analyte in the sample fluid. By using EIS to determine impedance of the electrochemical system, the respective analyte may be directly measured in the sample fluid (e.g., EIS may be label free), the excitation frequency may be selected to target a respective analyte, the analyte may not be consumed by a reaction, noise may be measured simultaneously to the response signal to improve the signal-to-noise ratio and evaluate the function of the sensor, and power consumption is reduces compared to other detection methods.

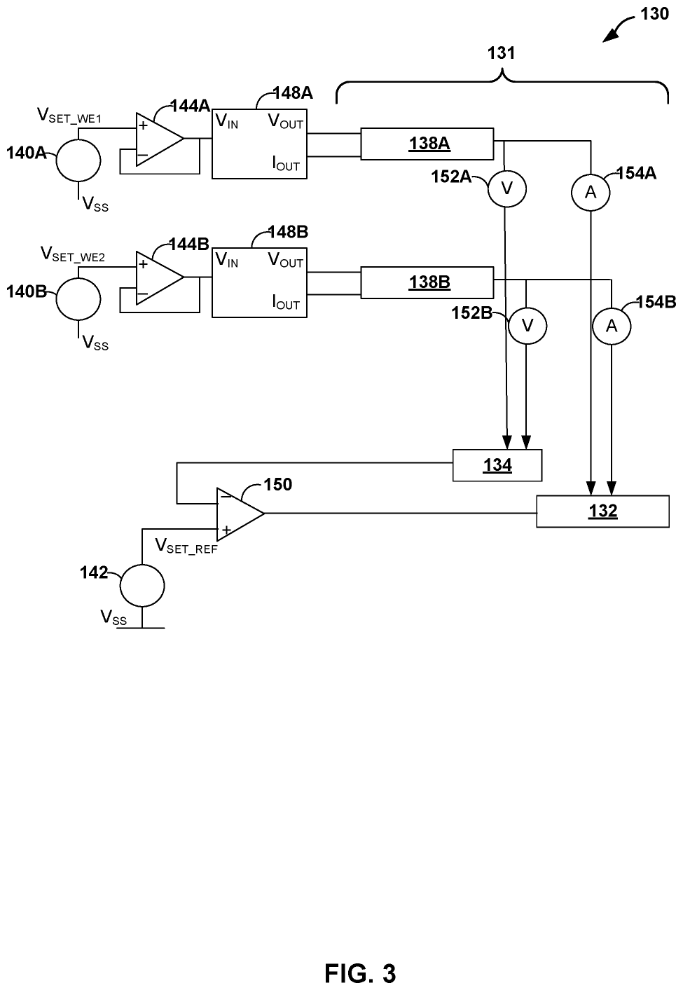

FIG. 3 is a schematic and conceptual partial circuit diagram illustrating an example medical device 130 that includes an electrochemical sensor 131 including a counter electrode 132, a common reference electrode 134, and a work platform having a plurality of respective work electrodes 138A and 138B (collectively, “work electrodes 138”) operatively coupled to corresponding electrical components, in accordance with one or more techniques of this disclosure. Electrochemical sensor 131 may be the same as or substantially similar to electrochemical sensor 10, illustrated in FIGS. 1, 2A, and 2B. Medical device 130 may be configured to detect the concentration of each of a plurality of analytes present in a sample fluid operatively coupled to (e.g., in fluid communication with) at least each respective work electrode of work electrodes 138. Counter electrode 132 may be configured to functions as a cathode when a respective work electrode of work electrodes 138 is operating as an anode and vice versa. Common reference electrode 134 may be configured to provide a stable and known electrode potential.

As discussed above, various signal processing techniques include applying a potential and/or current to a respective work electrode of work electrodes 138 and, in some examples, at least one of counter electrode 132 or common reference electrode 134. As illustrated in FIG. 3, medical device 130 may include a respective source supply voltage (V_{SS}) 140A and 140B (collectively, “work electrode source supply voltages 140”) operatively coupled to each respective work electrode of work electrodes 138. Each respective work electrode source supply voltage 140 may be operatively coupled to an amplifier 144A and 144B (collectively, “amplifiers 144”), which, in some examples, may be non-inverting amplifiers. In some examples, amplifiers 144 may reduce variation in the potential applied to the respective work electrode of work electrodes 138. The output of amplifiers 144 may be input to power electronics 148A and 148B (collectively, “power electronics 148”). A respective power electronics of power electronics 148 may be configured to supply a selected potential, a selected current, or both to a respective work electrode of work electrodes 138. In some examples, power electronics 148 may include a controller to, for example, overlay an AC excitation signal over a DC working potential. In some examples, power electronics 148 may include power conversion circuitry, such as an AC-to-direct-current (AC/DC) conversion device, a DC/DC conversion device, a buck conversion circuit, a boost conversion circuit, a buck-boost conversion circuit, a forward conversion circuit, a resonant-mode conversion circuit, a half-bridge circuit, an H-bridge circuit, and/or any other power conversion circuit. In some examples, power electronics 148 may include one or more switches configured to selectively supply power to a respective work electrode of work electrodes 138. By selecting a respective amplifier of amplifiers 144 and selecting a respective power electronics of power electronics 148, the potential and/or current delivered to a respective working electrode of work electrodes 138 may be controlled, for example, based on a selected signal processing technique for the respective work electrode. Additionally, or alternatively, selectively powering a respective work electrode of work electrodes 138 with one or more switches of power electronics 148 may enable dissipation of gradients, byproducts, or the like resulting from a first work electrode of work electrodes 138 before measuring with a second work electrode of work electrodes to reduce errors in the measurements of the second work electrode.

In some examples, a respective work electrode of work electrodes 138, e.g., an output of a respective work electrode of work electrodes 138, may be operatively coupled to a respective voltmeter of a plurality of voltmeters 152A and 152B (collectively, “voltmeters 152”). In some examples, a respective work electrode of work electrodes 138 may be operatively coupled to a respective ammeter of a plurality of ammeter 154A and 154B (collectively, “ammeter 154”). By operatively coupling work electrodes 138 to voltmeters 152 and ammeters 154, medical device 130 may measure the output potential and current of each respective work electrode of work electrodes 138.

In some examples, work electrodes 138 may be operatively coupled to common reference electrode 134. Common reference electrode 134 may provide a stable and known electrode potential to an inverting input of op amp 150. A source supply voltage (V_{SS}) 142 may be operatively coupled to a non-inverting input of op-amp 150. The output of op amp 150 may be operatively coupled to counter electrode 132.

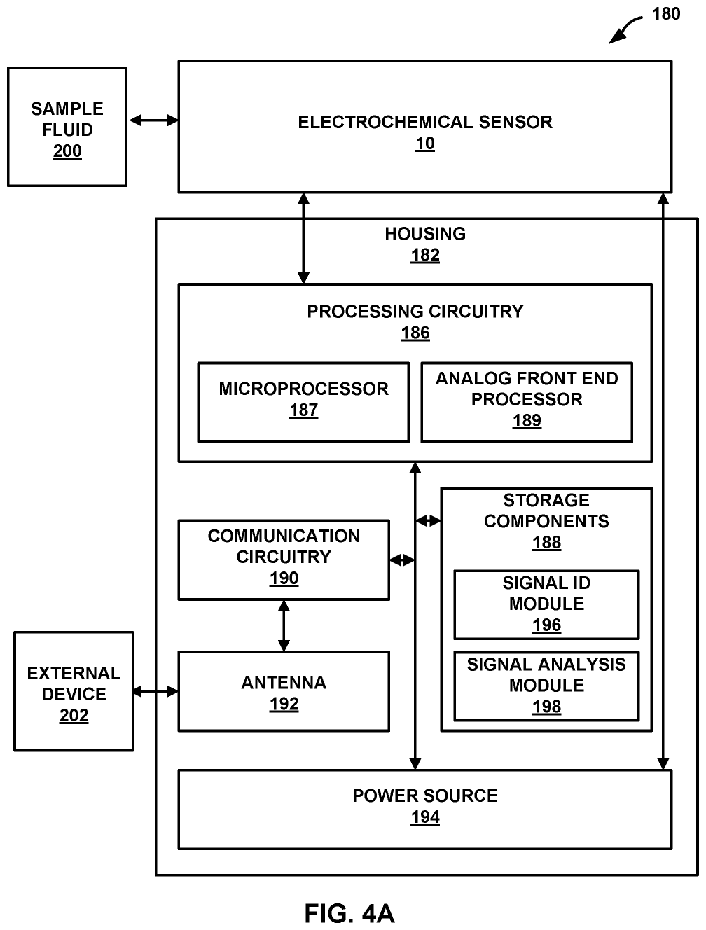

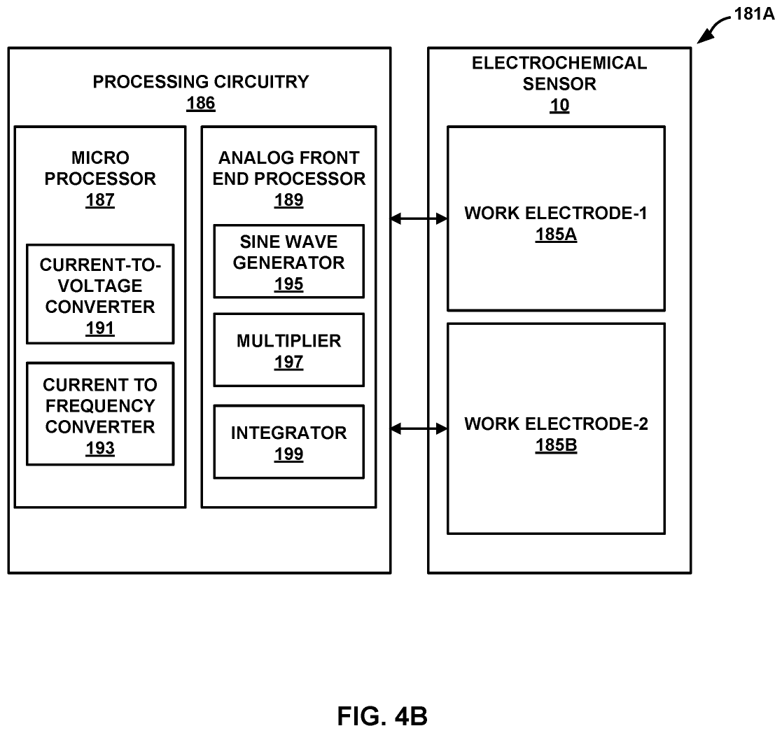

In some examples, an electrochemical sensor may be used in a medical device configured to be inserted within a patient, such as into the interstitial fluid of the patient. FIG. 4A is a schematic and conceptual block diagram illustrating an example medical device 180 configured to be inserted into the interstitial fluid of a patient. Medical device 180 may be the same as or substantially similar to medical device 130, illustrated in FIG. 3. Medical device 180 may include a housing 182, an electrochemical sensor 10, processing circuitry 186, storage components 188, communication circuitry 190, an antenna 192, and a power source 194. FIG. 4B is a schematic and conceptual block diagram illustrating an example configuration of medical device 181 having processing circuitry 186 and electrochemical sensor 10.

In some examples, at least a portion of a dielectric substrate (e.g., dielectric substrate 20) of the components of medical device 180 may define housing 102. In other examples, housing 102 may include a discrete material layer, for example, including but not limited to, a biocompatible coating, biocompatible casing, molded or 3-D printed plastics. Housing 182 may separate at least a portion of the components of medical device 180 including electrochemical sensor 10, processing circuitry 186, storage components 188, communication circuitry 190, an antenna 192, and a power source 194 from the environment surrounding medical device 180, e.g., sample fluid 200. In some examples, one or more components of medical device 180 may be disposed outside housing 182, such as, for example, affixed to an external surface of housing 182. For example, antenna 188 may be affixed to an external surface of housing 182 to improve transmission properties of antenna 192. Housing 182 may include any suitable shape, such as rectilinear or curvilinear. In some examples, housing 182 may be shaped to facilitate insertion of housing 182 into the interstitial fluid of a human patient. For example, housing 182 may include a circular shape to be loaded into an insertion tool or include rounded corners and edges to reduce irritation to the patient.

Housing 180 may be any suitable dimensions. In some examples, a height of housing 102 may be between approximately 1 millimeter and approximately 7 millimeters, such as approximately 2.35 millimeters. In some examples, a width of housing 182 may be between approximately 5 millimeters and approximately 15 millimeters, such as approximately 10.5 millimeters. In some examples, a length of the housing 182 may be between approximately 5 millimeters and approximately 15 millimeters, such as approximately 10.5 millimeters.

In some examples, at least a portion of electrochemical sensor 10 is fluidly coupled to the environment surrounding medical device 180. For example, at least a portion of a work electrode platform of electrochemical sensor 10 may be fluidly coupled to sample fluid 200. In some examples, housing 182 may include one or more apertures exposing at least a portion of electrochemical sensor 10 to sample fluid 200. In examples in which housing 182 includes a coating or a casing, electrochemical sensor 10 may protrude at least partially through a portion of housing 182.

Electrochemical sensor 10 may include a common reference electrode, a counter electrode, and a work electrode platform including a plurality of respective work electrodes 185A and 185B (collectively, “work electrodes 185”), illustrated in FIG. 4B. As discussed above, each respective work electrode of work electrodes 185 may be electrically coupled to the common reference electrode and, optionally, at least one counter electrode. Each respective work electrode of work electrodes 185 may include a respective reagent substrate configured to react with a selected analyte to produce a respective signal indicative of a concentration of the selected analyte. In some examples, electrochemical sensor 10 may include a dielectric substrate layer defining a first major surface and interconnect layer on first major surface and defining second major surface, where work electrodes 185 may be disposed on the second major surface and the interconnect layer electrically couples the common reference electrode and the at least one counter electrode to work electrodes 185.

Processing circuitry 186 may include various type of hardware, including, but not limited to, microprocessors, controllers, digital signal processors (DSPs), application specific integrated circuits (ASICs), field-programmable gate arrays (FPGAs), or equivalent discrete or integrated logic circuitry, as well as combinations of such components. The term “processing circuitry” may generally refer to any of the foregoing logic circuitry, alone or in combination with other logic circuitry, or any other equivalent circuitry. In some examples, processing circuitry 186 may represent and/or include additional components, such as sine wave generator 195, multiplier 197, integrators 199, current-to-voltage converter 191, current-to-frequency converter 193, or the like. Processing circuitry 186 represents hardware that can be configured to implement firmware and/or software that sets forth one or more of the algorithms described herein. For example, processing circuitry 186 may be configured to implement functionality, process instructions, or both for execution of processing instructions stored within one or more storage components 188, such as signal identification (ID) module 196 and/or signal analysis module 198.

Processing circuitry 186 is operatively coupled to electrochemical sensor 10 to receive from electrochemical sensor 10 a plurality of signals from work electrodes 185. Processing circuitry 186, e.g., via signal identification module 196, may be configured to identify a respective signal corresponding to a respective selected work electrode of work electrodes 185. For example, processing circuitry 186, e.g., via signal identification module 196, may include multiplexer 201 to identify a respective signal.

Processing circuitry, e.g., via signal analysis module 198, may be configured to process the identified signal to determine the concentration of the respective analyte associated with the respective selected work electrode, as discussed above, by amperometry, potentiometry, and/or EIS. In some examples, processing circuitry 186 may include an analog-to-digital converter communicatively coupled to a microprocessor 187. Microprocessor 187 may be configured to process a respective signal (e.g., converted by the analog-to-digital converter) corresponding to a respective selected work electrode of work electrodes 185 to determine the concentration of the respective analyte associated with the respective selected work electrode by amperometry or potentiometry.

In some examples, processing circuitry 186 may include an analog front end (AFE) processor 189 (e.g., an “AFE chip”). AFE processor 189 may be configured to process a respective signal corresponding to a respective selected work electrode of work electrodes 185 to determine the concentration of the respective analyte associated with the respective selected work electrode by amperometry, potentiometry, or EIS. In some examples, as illustrated in FIG. 4B, AFE processor 189 includes a dedicated input for each respective work electrode of work electrodes 185. In some such examples, AFE processor 189 may receive from each respective work electrode of work electrodes 185 a signal including a respective selected frequency for a selected analyte. The respective selected frequency for the selected analyte may be selected to increase a signal response for the selected analyte. For example, a selected frequency for glucose may include about 1000 Hz and a selected frequency for potassium may include about 600 Hz. In other examples, AFE processor 189 may receive a respective signal from each respective work electrode of work electrodes 185 including a respective frequency sweep (e.g., various frequencies ranging from about 1 Hz up to about 100 KHz). The frequency sweep may include at least one frequency or frequency range that results in a response signal from a selected analyte. In other examples, AFE processor 189 may be electrically connected to each work electrode of work electrodes 185 via a multiplexer (not depicted). In this way, medical device 180 may identify and process a plurality of signals, each respective signal of the plurality of signals corresponding to a respective work electrode of work electrodes 185. In other examples, e.g., particularly in examples in which the work electrode platform includes more than two work electrodes, AFE processor 189 may include a dedicated input for each respective work electrode of a first group of work electrodes from work electrodes 185 and may include an input electrically connected to a multiplexer (not depicted). The respective signals may include a respective selected frequency or a respective frequency sweep for each of the second group of work electrodes 185. In this way, some of work electrodes 185 may be interrogated directly while others may be interrogated in parallel via the multiplexer.

One or more storage components 188 may be configured to store information within medical device 180. One or more storage components 188, in some examples, include a computer-readable storage medium or computer-readable storage device. In some examples, one or more storage components 188 include a temporary memory, meaning that a primary purpose of one or more storage components 188 is not long-term storage. One or more storage components 188, in some examples, include a volatile memory, meaning that one or more storage components 188 does not maintain stored contents when power is not provided to one or more storage components 188. Examples of volatile memories include random access memories (RAM), dynamic random-access memories (DRAM), static random-access memories (SRAM), and other forms of volatile memories known in the art. In some examples, one or more storage components 188 are used to store program instructions for execution by processing circuitry 186. One or more storage components 188, in some examples, are used by software or applications running on processing circuitry 186 to temporarily store information during program execution.

In some examples, one or more storage components 188 may further include one or more storage components 188 configured for longer-term storage of information. In some examples, one or more storage components 188 include non-volatile storage elements. Examples of such non-volatile storage elements include flash memories, or forms of electrically programmable memories (EPROM) or electrically erasable and programmable (EEPROM) memories.

As noted above, storage components 188 may store signal identification module 196 and signal analysis module 198. Each of signal identification module 196 and signal analysis module 198 may be implemented in various ways. For example, one or more of signal identification module 196 and signal analysis module 198 may be implemented as an application or a part of an application executed by processing circuitry 186. In some examples, one or more of signal identification module 196 and signal analysis module 198 may be implemented as part of a hardware unit of medical device 180 (e.g., as circuitry). In some examples, one or more of signal identification module 196 and signal analysis module 198 may be implemented remotely on external device 202 as part of an application executed by one or more processors of external device 202 or as a hardware unit of external device 202.

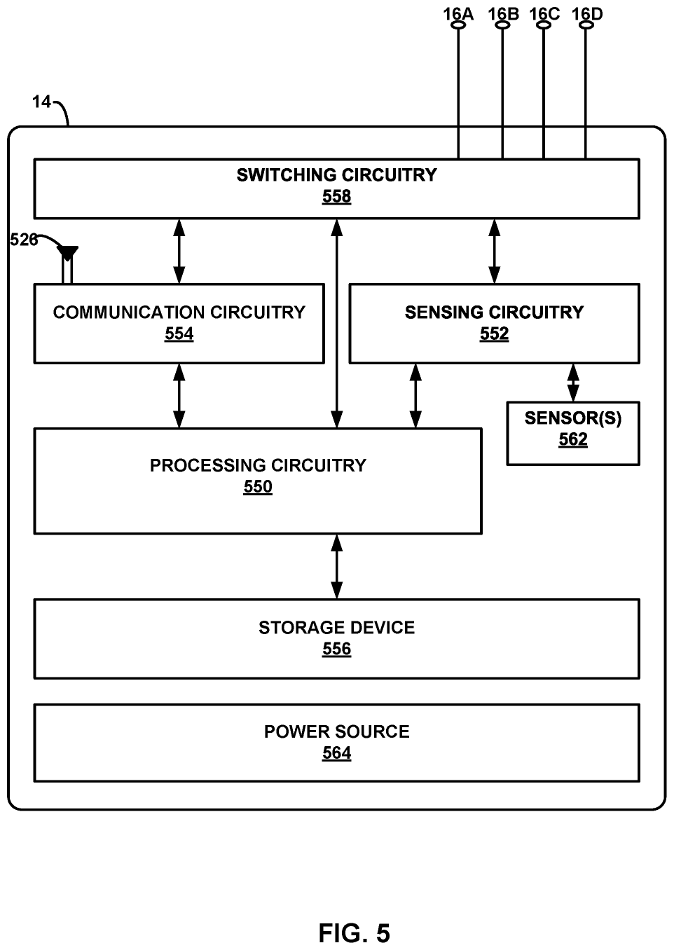

FIG. 5 is a functional block diagram illustrating an example configuration of IMD 14 of FIG. 1, in accordance with one or more techniques described herein. As seen in FIG. 5, IMD 14 includes electrodes 516A-516D (collectively, “electrodes 516”), antenna 526, processing circuitry 550, sensing circuitry 552, communication circuitry 554, storage device 556, switching circuitry 558, sensors 562, and power source 564.

Processing circuitry 550 may include fixed function circuitry and/or programmable processing circuitry. Processing circuitry 550 may include, for example, microprocessors, DSPs, ASICs, FPGAs, equivalent discrete or integrated logic circuitry, or a combination of any of the foregoing devices or circuitry. Accordingly, processing circuitry 550 may include any suitable structure, whether in hardware, software, firmware, or any combination thereof, to perform the functions ascribed herein to IMD 14.

Sensing circuitry 552 and communication circuitry 554 may be selectively coupled to electrodes 16 via switching circuitry 558, which may be controlled by processing circuitry 550. Sensing circuitry 552 may monitor signals from electrodes 516 in order to monitor electrical activity of heart (e.g., to produce an EGM), and/or subcutaneous tissue impedance, the impedance being indicative of at least some aspects of patient 4's cardiac activity and/or respiratory patterns. Electrodes 516 may be used to sense cardiac EGMs (e.g., cardiac ECGs) when IMD 14 is implanted in patient 4. In some examples, processing circuitry of IMD 14 may determine one or more patient-specific relationships between the cardiac EGM and one or more signals indicative of the concentration of one or more analytes. The cardiac EGMs may be stored in a memory of the IMD 14. In some examples, data derived from the EGMs may be transmitted via integrated antenna 526 to another medical device, such as external device 12.

Sensing circuitry 552 also may monitor signals from sensors 562, which may include light detectors, motion sensor(s), and any additional sensors that may be positioned on IMD 14. In some examples, sensing circuitry 552 may include one or more filters and amplifiers for filtering and amplifying signals received from one or more of electrodes 516 and/or sensor(s) 562. In some examples, sensing circuitry 552 may contain an analog to digital converter (ADC) for digitizing sensor signals.

Communication circuitry 554 may include any suitable hardware, firmware, software or any combination thereof for communicating with another device, such as external device 12 or another device or sensor, such as a pressure sensing device. Under the control of processing circuitry 550, communication circuitry 554 may receive downlink telemetry from, as well as send uplink telemetry to, external device 12 or another device with the aid of an internal or external antenna, e.g., antenna 526. In addition, processing circuitry 550 may communicate with a networked computing device via an external device (e.g., external device 12) and a computer network, such as the Medtronic CareLink® Network developed by Medtronic, Inc., of Minneapolis, Minnesota.

A clinician or other user may retrieve data from IMD 14 using external device 12, or by using another local or networked computing device configured to communicate with processing circuitry 550 via communication circuitry 554. The clinician may also program parameters of IMD 14 using external device 12 or another local or networked computing device.

In some examples, storage device 556 includes computer-readable instructions that, when executed by processing circuitry 550, cause IMD 14 and processing circuitry 550 to perform various functions attributed to IMD 14 and processing circuitry 550 herein. Storage device 556 may include one or both of a short-term memory or a long-term memory. The memory may include, for example, RAM, DRAM, SRAM, magnetic discs, optical discs, flash memories, or forms of EPROM or EEPROM. In some examples, the memory is used to store program instructions for execution by processing circuitry 550.

Power source 564 is configured to deliver operating power to the components of IMD 14. Power source 564 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 564 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

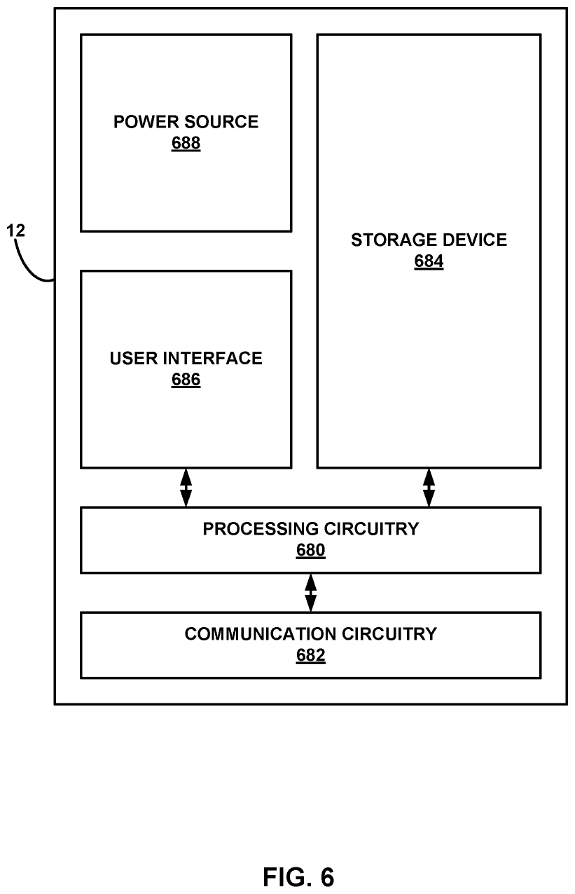

FIG. 6 is a block diagram illustrating an example configuration of components of external device 12 of FIG. 1, in accordance with one or more techniques of this disclosure. In the example of FIG. 5, external device 12 includes processing circuitry 680, communication circuitry 682, storage device 684, user interface 686, and power source 688.

Processing circuitry 680 may include fixed function circuitry and/or programmable processing circuitry. Processing circuitry 680 may include, for example, microprocessors, DSPs, ASICs, FPGAs, equivalent discrete or integrated logic circuitry, or a combination of any of the foregoing devices or circuitry. Accordingly, processing circuitry 680 may include any suitable structure, whether in hardware, software, firmware, or any combination thereof, to perform the functions ascribed herein to external device 12.

Communication circuitry 682 may include any suitable hardware, firmware, software or any combination thereof for communicating with another device, such as IMD 14 and/or medical device 180. Under the control of processing circuitry 680, communication circuitry 682 may receive downlink telemetry from, as well as send uplink telemetry to, IMD 14, medical device 180, or another device.

In some examples, storage device 684 includes computer-readable instructions that, when executed by processing circuitry 680, cause external device 12 and processing circuitry 680 to perform various functions attributed to IMD 14, medical device 180 and/or processing circuitry 680 herein. Storage device 684 may include one or both of a short-term memory or a long-term memory. The memory may include, for example, RAM, DRAM, SRAM, magnetic discs, optical discs, flash memories, or forms of EPROM or EEPROM. In some examples, the memory is used to store program instructions for execution by processing circuitry 680. Storage device 684 may be used by software or applications running on external device 12 to temporarily store information during program execution.

Data exchanged between external device 12 and IMD 14 may include an EGM signal or characteristics. External device 12 may transmit data including computer readable instructions which, when implemented by IMD 14, may control IMD 14 to export collected data. (e.g., data corresponding to a cardiac EGM signal) to external device 12. In turn, external device 12 may receive the collected data from IMD 14 and store the collected data in storage device 684. Data exchanged between external device 12 and medical device 180 may include one or more signals indicative of a concentration of one or more analytes in a patient. The data may also include values for concentrations of the one or more analytes in the patient. External device 12 may transmit data including computer readable instructions which, when implemented by medical device 180, may control medical device 180 to export collected data. (e.g., data corresponding to concentrations of one or more analytes) to external device 12. In turn, external device 12 may receive the collected data from medical device 180 and store the collected data in storage device 684.

A user, such as a clinician or patient 4, may interact with external device 12 through user interface 686. User interface 686 includes a display (not shown), such as an LCD or LED display or other type of screen, with which processing circuitry 680 may present information related to IMD 14 (e.g., EGM signals obtained from at least one electrode or at least one electrode combination). In addition, user interface 686 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 680 of external device 12 and provide input. In other examples, user interface 686 also includes audio circuitry for providing audible notifications, instructions or other sounds to patient 4, receiving voice commands from patient 4, or both. Storage device 684 may include instructions for operating user interface 686 and for managing power source 688.

Power source 688 is configured to deliver operating power to the components of external device 12. Power source 688 may include a battery and a power generation circuit to produce the operating power. In some examples, the battery is rechargeable to allow extended operation. Recharging may be accomplished by electrically coupling power source 688 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

External device 12 and/or processing circuitry 680 thereof may be configured to perform the techniques described herein. For example, external device 12 may receive an EGM signal and/or EGM characteristics from an IMD (e.g., IMD 14). External device 12 may also receive one or more signals indicative of a concentration of one or more analytes in a patient from a medical device (e.g., medical device 180), and or may receive values for concentrations of the one or more analytes in the patient from the medical device. Processing circuitry 680 may determine one or more patient-specific relationships between the one or more signals indicative of the concentration of the one or more analytes over a first period of time and the cardiac EGM for the patient collected over the first period of time, and, based on the patient-specific relationships, determine an estimated concentration of the one or more analytes based on the EGM for the patient collected over a second period of time. In order to determine the one or more patient-specific relationships between the one or more signals and the cardiac EGM, processing circuitry 680 may be configured to train a machine learning model to determine the estimated concentration of the one or more analytes as output based on an input cardiac EGM segment using a set of training data that includes the one or more signals indicative of the concentration of the one or more analytes and the cardiac EGM, wherein each segment of the one or more signals includes a label indicating a corresponding segment of the cardiac EGM.

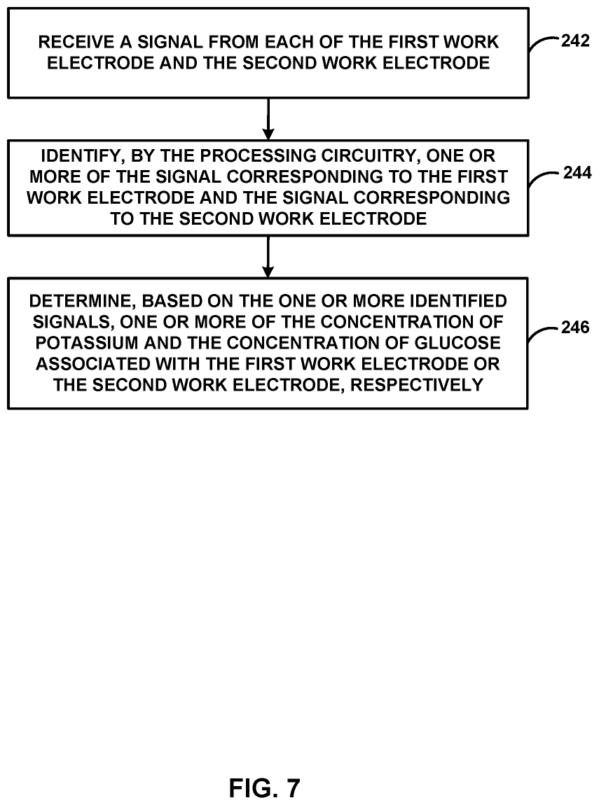

FIG. 7 is a flow chart illustrating an example operation for determining an analyte concentration, in accordance with one or more techniques of this disclosure. Although the technique illustrated in FIG. 7 will be described with respect to medical device 180 illustrated in FIG. 4A including electrochemical sensor 10 of FIGS. 1, 2A, 2B, 4A, and 4B, in some examples, the technique illustrated in FIG. 7 may use other medical devices or other electrochemical sensors to determine a concentration of an analyte, including, but not limited to, medical device 130 of FIG. 3, IMD 14 of FIG. 1, or external device 12 of FIG. 6.

Electrochemical sensor 10 includes counter electrode 82, common reference electrode 84, and work electrode platform 16 (FIG. 1). Work electrode platform 16 includes one or more work electrodes, e.g., two work electrodes. In some examples, a first work electrode may include a first reagent substrate configured to react with potassium to produce a signal (e.g., a current and/or a potential) indicative of a concentration of potassium, and a second work electrode may include a second reagent substrate configured to react with glucose to produce a signal (e.g., a current and/or a potential) indicative of a concentration of glucose. The work electrodes of work electrode platform 16 may be electrically coupled to counter electrode 82 and common reference electrode 84.

Processing circuitry of system 2, e.g., processing circuitry 186 of medical device 180, processing circuitry of electrochemical sensor 10, processing circuitry 550 of IMD 14, and/or processing circuitry 680 of external device 12, may be configured to perform the techniques of FIG. 7. Processing circuitry of system 2 may receive a signal from each of the first work electrode and the second work electrode (242). In some examples, the plurality of signals may include conditioned signals, unconditioned signals, or both. For example, a portion of the signals may include analogue signals and a portion of the signals may include digital signals.

Processing circuitry of system 2 may identify one or more of a signal corresponding to the first work electrode and a signal corresponding to the second work electrode (244). In some examples, identifying the respective signal of the plurality of signals may include using timing signals to associate the respective signal with a selected work electrode of work electrodes 88. In some examples, identifying the respective signal of the plurality of signals may include using a multiplexer to associate the respective signal with a selected work electrode of work electrodes 88. By identifying the respective signal associated with a selected work electrode of work electrodes 88, processing circuitry of system 2 may determine an appropriate technique to process the respective signal. Processing circuitry of system 2 may determine, based on the identified signal(s), one or more of the concentrations of the one or more analytes, e.g., the concentration of potassium and the concentration of glucose, associated with the first work electrode or the second work electrode, e.g., respective work electrodes of work electrodes 88 of work electrode platform 16, respectively (246). For example, processing circuitry of system 2 may process the identified signal by at least one of amperometry, potentiometry, or EIS. In some examples, the identified signal may include a current, such as change in current between the respective work electrode of work electrodes 88 and common reference electrode 84, such that processing circuitry of system 2 may determine a concentration of the respective analyte based at least in part on amperometry, as discussed above. In some examples, the identified signal may include a potential, such as a difference in the potential between the respective work electrode of work electrodes 88 and common reference electrode 84, such that processing circuitry of system 2 may determine a concentration of the respective analyte based at least in part on potentiometry, as discussed above. In some examples, the identified signal may include a response signal, such as a current between the respective work electrode of work electrodes 88 and counter electrode 82 in response to an excitation signal, such that processing circuitry of system 2 may determine a concentration of the respective analyte base at least in part on EIS, as discussed above.

In some examples, the technique illustrated in FIG. 7 may be performed while medical device 180 is disposed within a biological system, such as inserted within an interstitial fluid of a human patient.

In some examples, the technique illustrated in FIG. 7 optionally includes transmitting, by antenna 192 operatively coupled to processing circuitry 186, the determined concentration of the respective analyte to one or more of external device 12 or IMD 14. In some examples, external device 12 may be located outside of the biological system, such as outside of the interstitial fluid of a human patient. Additionally, or alternatively, processing circuitry of external device 12 may perform the techniques illustrated in FIG. 7, and processing circuitry 186 of medical device 180 may control communication circuitry 190 to transmit, by antenna 192, the signals indicative of the glucose and potassium concentrations to external device 12. Additionally, or alternatively, processing circuitry of IMD 14 may perform the techniques illustrated in FIG. 7, and processing circuitry 186 of medical device 180 may control communication circuitry 190 to transmit, by antenna 192, the signals indicative of the glucose and potassium concentrations to IMD 14.

Although the techniques of FIG. 7 are primarily described with reference to two work electrodes associated with determining a glucose concentration and a potassium concentration, the techniques of FIG. 7 can be performed with more than two work electrodes, e.g., five work electrodes, seven work electrodes, or 10 electrodes. Additionally, while glucose concentrations and potassium concentrations can advantageously serve as indicators of patient disease state, e.g., diabetes, and associated comorbidities, e.g., heart failure and/or kidney failure, different combinations of analytes are also possible, e.g., a combination of creatinine, glucose, and potassium, as described herein.

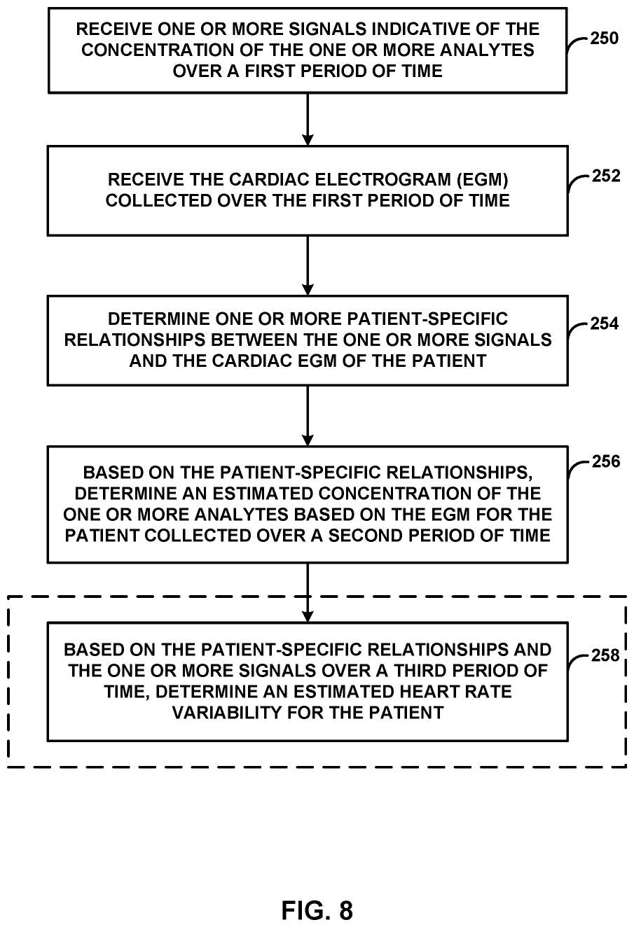

FIG. 8 is a flow chart illustrating an example operation for determining an estimated concentration of an analyte based on cardiac electrogram (EGM) data, in accordance with one or more techniques of this disclosure. Processing circuitry of system 2, e.g., processing circuitry 186 of medical device 180, processing circuitry of electrochemical sensor 10, processing circuitry XX of IMD 14, and/or processing circuitry XX of external device 12, may be configured to perform the techniques of FIG. 8. Processing circuitry of system 2 may receive one or more signals indicative of the concentrations of one or more analytes, e.g., potassium and glucose, over a first period of time, e.g., one minute, one hour, one day, one week, one month, or two months (250). Processing circuitry of system 2 may also receive a cardiac EGM signal collected over the first period of time (252). In some examples, IMD 14 collects the cardiac EGM. Processing circuitry of system 2 may determine one or more patient-specific relationships between the one or more signals indicative of the concentrations of glucose and potassium and the cardiac EGM of the patient (254). In some examples, the cardiac EGM includes an ECG. In examples in which the cardiac EGM includes an ECG, processing circuitry of system 2 may determine the one or more patient-specific relationships between the one or more signals indicative of the concentrations of the one or more analytes and characteristics of the ECG. Characteristics of the ECG may include one or more of: an RT interval, a QT interval, T-wave amplitude, T-wave inversion, U-wave visibility, ST segment depression, R-wave amplitude, or QRS width.

Determining one or more patient-specific relationships may include determining typical or baseline ECG characteristics and identifying relative changes in the characteristics over a time period. The processing circuitry may also determine typical or baseline analyte concentrations and relative changes in the analyte concentrations over the same time period. As an example, changes (e.g., increases or decreases) in the RT interval of patient 4 from a patient baseline may be indicative of changes, e.g., increases or decreases, in glucose concentration. Typical ranges of RT interval and/or QT interval, e.g., 0.35 seconds-0.43 seconds, may be indicative of normal glucose concentrations. As another example, changes in ECG characteristics such as one or more of: flattening of T-waves, i.e., decreasing T-wave amplitude, inversion of T-waves, QT interval prolongation, U-wave visibility, and/or ST segment depression may be indicative of hypokalemia, i.e., low potassium concentration, at varying levels of severity. As another examples, changes in ECG characteristics such as one or more of: relatively tall, peaked T-waves with a relatively narrow base, i.e., increased T-wave amplitude, prolonged PR intervals, disappearing or flattening P-wave, widening of the QRS, i.e., increasing QRS interval, and/or increased R-wave amplitude, may be indicative of hyperkalemia, i.e., high potassium concentration, at varying levels of severity. Typical ranges of PR interval, e.g., 0.12 seconds-0.20 seconds, QRS width, e.g., 0.08 seconds-0.12 seconds, and/or QT interval, e.g., 0.35 seconds-0.43 seconds, may be indicative of normal potassium concentrations. Processing circuitry of system 2 may save the patient-specific relationships to a database in memory (e.g., storage components 188, storage device 684, and/or storage device 556), in which ECG characteristics are associated with corresponding analyte concentrations or changes. In this way, processing circuitry of system 2 may build a database of ECG characteristics correlated with analyte concentrations and/or changes in analyte concentrations for the patient.

In some examples, processing circuitry of system 2 may implement one or more machine learning models to determine the one or more patient-specific relationships between the one or more signals indicative of the one or more analyte concentrations, e.g., glucose concentration and potassium concentration, and the cardiac EGM signal, e.g., ECG signal. The one or more machine learning models may include, as examples, neural networks, such as deep neural networks, which may include convolutional neural networks, multi-layer perceptrons, and/or echo state networks, as examples. In some examples, processing circuitry of system 2 may train the one or more machine learning models to accept EGM signals as input, and output one or more analyte concentrations or changes in analyte concentrations. In some examples, processing circuitry of system 2 may train the one or more machine learning models to accept one or more analyte concentrations or changes in analyte concentrations as input, and output an estimated EGM signal, or estimated change to a characteristic in an EGM signal. Processing circuitry of system 2 may be configured to generate a set of training data including one or more analyte signals received over a period of time and an EGM signal received over the same period of time. For example, processing circuitry of system 2 may receive a signal indicative of the concentration of glucose in patient 4 and a signal indicative of the concentration of potassium in patient 4 over a first time period. Processing circuitry of system 2 may also receive an ECG signal for patient 4 collected over the first time period and save the ECG signal to the database in memory. The processing circuitry may save the signals indicative of the concentrations of glucose and potassium to the database in memory with associated datapoints of the signals to corresponding datapoints of the ECG received at the same time (within a margin of error of the measurement instruments) as the set of training data. In some examples, processing circuitry of system 2 may continuously train the one or more machine learning models based on analyte signals and ECG signals received during a monitoring period.

In some examples, processing circuitry of system 2 may save each segment of the signals indicative of the potassium and glucose concentrations with a label indicating a corresponding segment of the ECG signal. The one or more machine learning models may be implemented using any number of models for supervised and/or reinforcement learning, such as, but not limited to, an artificial neural network, a decision tree, a naïve Bayes network, a support vector machine, or a k-nearest neighbor model. Processing circuitry of system 2 may train a machine learning model based on the set of training data to accept the signals indicative of the concentrations of glucose and potassium as input and output an estimated ECG signal. In some examples, processing circuitry of system 2 may train a machine learning model based on the set of training data to accept the ECG signal as input, and output signals indicative of the concentrations of glucose and potassium as output. In some examples, rather than using the raw signals indicative of glucose and potassium concentrations, processing circuitry of system 2 may use determined concentrations of glucose and potassium to train the one or more machine learning models. In some examples, processing circuitry of system 2 may train a first machine learning model to output an estimated ECG signal based on a glucose concentration or signal as input and may train a second machine learning model to output an estimated ECG signal based on a potassium concentration or signal as input. In some examples, processing circuitry of system 2 may train a third machine learning model to output an estimated glucose concentration or signal based on an ECG signal as input. In some examples, processing circuitry of system 2 may train a fourth machine learning model to output an estimated potassium concentration or signal based on an ECG signal as input.

Processing circuitry of system 2 may compare a prediction of classification by the one or more machine learning models to a target output, e.g., processing circuitry of system 2 may compare the predicted estimation of the glucose and/or potassium concentrations based on the ECG signal to the concentrations of the glucose and/or potassium concentrations based on the signals indicative of the glucose and potassium concentrations. Based on an error in the estimation of the glucose and/or potassium concentrations, processing circuitry of system 2 implementing the one or more machine learning models may send or apply a modification to weights of the one or more machine learning models or otherwise modify/update the machine learning model.

Processing circuitry of system 2 may periodically or continuously update the training set of data with new patient data outside the original training dataset. In this way, the one or more machine learning models may be continuously updated, which may continuously improve the system and the accuracy of outputs of the one or more machine learning models.

Based on the patient-specific relationships, processing circuitry of system 2 (e.g., processing circuitry 186 of medical device 180, processing circuitry of electrochemical sensor 10, processing circuitry 550 of IMD 14, and/or processing circuitry 680 of external device 12) may determine an estimated concentration of the glucose and potassium based on the ECG signal for the patient collected over a second period of time, e.g., months to years (256). In some examples, processing circuitry of system 2 may determine an estimated concentration of glucose and/or potassium based on a database saved in memory correlating an ECG signal to estimated concentrations of glucose and/or potassium. In some examples, processing circuitry of system 2 may implement one or more machine learning models to determine the estimated concentration of the glucose and/or potassium based on the cardiac EGM signal over the second period of time. Processing circuitry of system 2 may apply the cardiac EGM signal as input to the one or more machine learning models, and generate, as output of the one or more machine learning models, one or more signals indicative of the potassium and/or glucose concentrations. In some examples, the one or more machine learning models may be configured to identify features of the cardiac EGM, e.g., characteristics of an ECG signal. The one or more machine learning models may be configured to output values indicative of one or more patient-specific relationships and/or may output values indicative of the estimated concentration of potassium and/or the estimated concentration of glucose based on the cardiac EGM data, based on the patient-specific relationships.

Optionally, processing circuitry of system 2 may, based on the patient-specific relationships and the one or more signals produced over a third period of time, determine an estimated heart rate variability for the patient (258). Changes in heart rate variability may be indicative of changes in the disease state of patient 4. For example, changes in glucose and/or potassium concentration are associated with changes in cardiac action potential duration. Changes in cardiac action potential can cause changes in heart rate variability. Consequently, processing circuitry may determine an estimate of heart rate variability based on the signals indicative of glucose and/or potassium concentration described herein.

Changes in heart rate variability are associated with atrial fibrillation and heart failure (HF). In some examples, changes in heart rate variability may be indicative of kidney failure. In examples in which patient 4 is diabetic, the techniques of this disclosure may enable a user to monitor for signs of comorbidities, such as HF and/or kidney failure. In this way, the user, e.g., the patient or a clinician, may identify and/or address changes in patient disease state relatively early, which may improve patient outcomes.

Although described primarily in the context of training the one or more machine learning models with patient data, in some examples, processing circuitry of system 2 may train the one or more machine learning models with aggregate patient data instead of or in addition to the patient data to determine one or more of: one or more relationships between the one or more signals indicative of the glucose concentration and the potassium concentration and the characteristics of the ECG, an estimated glucose concentration and/or an estimated potassium concentration, an estimated heart rate variability, and/or a patient disease state or a change in patient disease state.

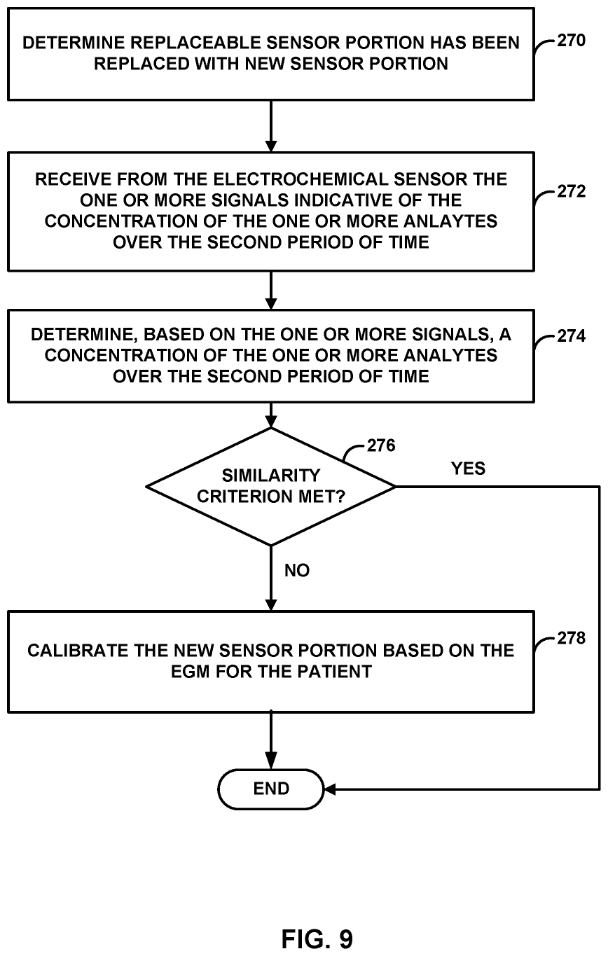

FIG. 9 is a flow chart illustrating an example operation for calibrating a new sensor portion based on cardiac EGM data, in accordance with one or more techniques of this disclosure. In some examples, medical device 180 may include a replaceable portion, e.g., a replaceable electrochemical sensor 10 portion. In some examples, the user may want to confirm a new electrochemical sensor portion of, for example, medical device 180 (FIG. 4) is functioning and outputting accurate analyte concentrations. Processing circuitry of system 2, e.g., processing circuitry 186 of medical device 180, processing circuitry of electrochemical sensor 10, processing circuitry of IMD 14, and/or processing circuitry of external device 12, may be configured to perform the techniques of FIG. 9.

Processing circuitry of system 2 may determine the replaceable electrochemical sensor portion has been replaced with a new electrochemical sensor portion (270). In some examples, processing circuitry of system 2 may receive user input, e.g., via a user interface of external device 12, indicating that the replaceable electrochemical sensor portion has been replaced with the new electrochemical sensor portion. In other examples, processing circuitry of system 2 may determine the sensor portion has been replaced based on the signal data, e.g., based on an interruption in signal data. In some examples, processing circuitry of system 2 may determine that the sensor portion has been replaced based on identification information associated with the sensor portion hardware, e.g., that an identification number of the sensor portion has changed. Processing circuitry of system 2 may receive, from the new electrochemical sensor portion, one or more signals indicative of the concentration of the one or more analytes, e.g., glucose and potassium, over the second period of time (272). Processing circuitry of system 2 may determine, based on the signals indicative of the glucose concentration and the potassium concentration, the glucose concentration and the potassium concentration over the second period of time (274). Processing circuitry of system 2 may compare the glucose concentration and the potassium concentration to the estimated glucose concentration and the estimated potassium concentration determined based on the cardiac EGM and the patient-specific relationships. If a similarity criterion is met (“YES” of 276), the process ends, and the new electrochemical sensor portion is deemed to be accurate. In some examples, determining whether the similarity criterion is met comprises determining if a difference between the glucose and potassium concentrations determined based on the signals indicative of the glucose and potassium concentrations and the estimated glucose and potassium concentrations based on the cardiac EGM and patient-specific relationships falls below a threshold similarity value, e.g., 10% different. In some examples, determining whether the similarity criterion is met comprises determining if a correlation (e.g., a correlation coefficient) of the glucose and potassium concentrations determined based on the signals indicative of the glucose and potassium concentrations and the estimated glucose and potassium concentrations based on the cardiac EGM and patient-specific relationships is statistically significant (e.g., the correlation coefficient is 0.7 or greater). If the similarity criterion is not met (“NO” of 276), processing circuitry of system 2 calibrates the new electrochemical sensor portion based on the cardiac EGM for the patient (278).

The following clauses include example subject matter of the present disclosure.

Example 1. A system comprising: an electrochemical sensor comprising one or more work electrodes configured to produce one or more signals indicative of a concentration of one or more analytes in a patient; an implantable medical device (IMD) configured to sense a cardiac electrogram (EGM) of the patient; and processing circuitry in communication with the electrochemical sensor and the IMD, wherein the processing circuitry is configured to: receive from the electrochemical sensor the one or more signals indicative of the concentration of the one or more analytes over a first period of time; receive from the implantable medical device the cardiac EGM collected over the first period of time; determine one or more patient-specific relationships between the one or more signals and the cardiac EGM for the patient, based on the received one or more signals and the collected cardiac EGM over the first period of time; and based on the patient-specific relationships, determine an estimated concentration of the one or more analytes based on the EGM for the patient collected over a second period of time.

Example 2. The system of example 1, further comprising a biocompatible medical device comprising the electrochemical sensor and configured for insertion through skin and into interstitial fluid of the patient.

Example 3. The system of example 1, wherein the electrochemical sensor includes a common counter electrode; a common reference electrode; and a work electrode platform comprising the one or more work electrodes, wherein the one or more work electrodes include: a first work electrode comprising a first reagent substrate configured to react with potassium to produce a signal indicative of a concentration of potassium; and a second work electrode comprising a second reagent substrate configured to react with glucose to produce a signal indicative of a concentration of glucose, wherein the first work electrode and the second work electrode are electrically coupled to the common counter electrode and common reference electrode, and wherein the processing circuitry is further configured to: receive from the electrochemical sensor a signal from each of the first work electrode and the second work electrode; identify one or more of a signal corresponding to the first work electrode and a signal corresponding to the second work electrode; and determine, based on the identified signal, one or more of the concentration of potassium and the concentration of glucose associated with the first work electrode or the second work electrode, respectively.

Example 4. The system of example 1, wherein the electrochemical sensor includes a replaceable sensor portion comprising one or more work electrodes, and wherein the processing circuitry is further configured to: determine the replaceable sensor portion has been replaced with a new sensor portion; receive from the electrochemical sensor the one or more signals indicative of the concentration of the one or more analytes over the second period of time; determine, based on the one or more signals, a concentration of the one or more analytes over the second period of time; determine, based on the patient-specific relationships, that the concentration does not satisfy a threshold similarity with the estimated concentration of the one or more analytes; and calibrate the new sensor portion based on the EGM for the patient collected over the second period of time.

Example 5. The system of example 1, wherein the cardiac EGM includes an electrocardiogram (ECG), and wherein the processing circuitry is configured to determine the one or more patient-specific relationships between the one or more signals and characteristics of the ECG, wherein the characteristics include one or more of: an RT interval, a QT interval, T-wave amplitude, T-wave inversion, U-wave visibility, ST segment depression, R-wave amplitude, or QRS width.

Example 6. The system of example 1, wherein to determine the one or more patient-specific relationships between the one or more signals and the cardiac EGM, the processing circuitry is configured to: create a set of training data that includes the one or more signals produced over the first period of time indicative of the concentration of the one or more analytes and the cardiac EGM collected over the first period of time, wherein each segment of the one or more signals includes a label indicating a corresponding segment of the cardiac EGM; train a machine learning model to determine the estimated concentration of the one or more analytes as output based on an input cardiac EGM segment.

Example 7. The system of example 1, wherein each work electrode of the one or more work electrodes includes a respective membrane disposed on a respective reagent substrate, and wherein the respective membrane is selectively permeable to the respective analyte.

Example 8. The system of example 1, wherein the first period of time includes a week.

Example 9. The system of example 1, wherein the processing circuitry is further configured to determine, based on the patient-specific relationships and the one or more signals produced over a third period of time, an estimated heart rate variability for the patient.

Example 10. A method comprising: producing, by an electrochemical sensor comprising one or more work electrodes, one or more signals indicative of a concentration of one or more analytes in a patient; sensing, by an implantable medical device (IMD), a cardiac electrogram (EGM) of the patient; receiving from the electrochemical sensor, by processing circuitry in communication with the electrochemical sensor and the IMD, one or more signals indicative of the concentration of the one or more analytes over a first period of time; receiving from the IMD, by the processing circuitry, the cardiac EGM collected over the first period of time; determining, by the processing circuitry, one or more patient-specific relationships between the one or more signals and the cardiac EGM for the patient, based on the received one or more signals and the collected cardiac EGM over the first period of time; and based on the patient-specific relationships, determining, by the processing circuitry, an estimated concentration of the one or more analytes based on the EGM for the patient collected over a second period of time.

Example 11. The method of example 10, wherein a biocompatible medical device includes the electrochemical sensor and is configured for insertion through skin and into interstitial fluid of the patient.

Example 12. The method of example 10, further comprising: from the electrochemical sensor, receiving, by the processing circuitry, a signal from each of a first work electrode comprising a first reagent substrate configured to react with potassium to produce a signal indicative of a concentration of potassium and a second work electrode comprising a second reagent substrate configured to react with glucose to produce a signal indicative of a concentration of glucose, wherein the first work electrode and the second work electrode are electrically coupled to a common counter electrode and a common reference electrode; identifying, by the processing circuitry, one or more of a signal corresponding to the first work electrode and a signal corresponding to the second work electrode; and based on the identified signal, determining, by the processing circuitry, one or more of a concentration of potassium and a concentration of glucose associated with the first work electrode or the second work electrode, respectively.

Example 13. The method of example 10, further comprising: determining, by the processing circuitry, that a replaceable portion of the electrochemical sensor comprising one or more work electrodes has been replaced with a new sensor portion; from the electrochemical sensor, receiving, by the processing circuitry, the one or more signals indicative of the concentration of the one or more analytes over the second period of time; based on the one or more signals, determining, by the processing circuitry, a concentration of the one or more analytes over the second period of time; based on the patient-specific relationships, determining, by the processing circuitry, that the concentration does not satisfy a threshold similarity with the estimated concentration of the one or more analytes; and calibrating, by the processing circuitry, the new sensor portion based on the EGM for the patient collected over the second period of time.

Example 14. The method of example 10, wherein the cardiac EGM includes an electrocardiogram (ECG).

Example 15. The method of example 14, wherein determining the one or more patient-specific relationships includes: determining, by the processing circuitry, relationships between the one or more signals and characteristics of the ECG, wherein the characteristics include one or more of: an RT interval, a QT interval, T-wave amplitude, T-wave inversion, U-wave visibility, ST segment depression, R-wave amplitude, or QRS width.

Example 16. The method of example 10, wherein determining the one or more patient-specific relationships between the one or more signals and the cardiac EGM includes: creating, by the processing circuitry, a set of training data that includes the one or more signals produced over the first period of time indicative of the concentration of the one or more analytes and the cardiac EGM collected over the first period of time, wherein each segment of the one or more signals includes a label indicating a corresponding segment of the cardiac EGM; and training, by the processing circuitry, a machine learning model to determine the estimated concentration of the one or more analytes as output based on an input cardiac EGM segment.

Example 17. The method of example 10, wherein each work electrode of the one or more work electrodes includes a respective membrane disposed on a respective reagent substrate, and wherein the respective membrane is selectively permeable to the respective analyte.

Example 18. The method of example 10, wherein the first period of time includes a week.

Example 19. The method of example 1, further comprising: based on the patient-specific relationships and the one or more signals produced over a third period of time, determining, by the processing circuitry, an estimated heart rate variability for the patient.

Example 20. A non-transitory computer-readable storage medium, comprising instructions that, when executed by processing circuitry, cause the processing circuitry to: receive from an electrochemical sensor, one or more signals indicative of a concentration of a one or more analytes over a first period of time; receive from an implantable medical device a cardiac electrogram (EGM) collected over the first period of time; determine one or more patient-specific relationships between the one or more signals and the cardiac EGM for a patient, based on the received one or more signals and the collected cardiac EGM over the first period of time; and based on the patient-specific relationships, determine an estimated concentration of the one or more analytes based on the EGM for the patient collected over a second period of time.

Various examples have been described. These and other examples are within the scope of the following claims.

What is claimed is:

1. A system comprising:
an electrochemical sensor comprising one or more work electrodes configured to produce one or more signals indicative of a concentration of one or more analytes in a patient;
an implantable medical device (IMD) configured to sense a cardiac electrogram (EGM) of the patient; and
processing circuitry in communication with the electrochemical sensor and the IMD, wherein the processing circuitry is configured to:
determine one or more patient-specific relationships between the one or more signals indicative of the concentration of the one or more analytes over a first period of time and the cardiac EGM for the patient collected over the first period of time; and
based on the patient-specific relationships, determine an estimated concentration of the one or more analytes based on the EGM for the patient collected over a second period of time.

2. The system of claim 1, further comprising a biocompatible medical device comprising the electrochemical sensor and configured for insertion through skin and into interstitial fluid of the patient.

3. The system of claim 1,
wherein the electrochemical sensor comprises:
a common counter electrode;
a common reference electrode; and
a work electrode platform comprising the one or more work electrodes, wherein the one or more work electrodes comprise:
a first work electrode comprising a first reagent substrate configured to react with potassium to produce a signal indicative of a concentration of potassium; and
a second work electrode comprising a second reagent substrate configured to react with glucose to produce a signal indicative of a concentration of glucose,
wherein the first work electrode and the second work electrode are electrically coupled to the common counter electrode and common reference electrode, and wherein the processing circuitry is further configured to:
receive from the electrochemical sensor a signal from each of the first work electrode and the second work electrode;
identify one or more of a signal corresponding to the first work electrode and a signal corresponding to the second work electrode; and
determine, based on the identified signal, one or more of the concentration of potassium and the concentration of glucose associated with the first work electrode or the second work electrode, respectively.

4. The system of claim 1, wherein the electrochemical sensor comprises a replaceable sensor portion comprising one or more work electrodes, and wherein the processing circuitry is further configured to:
determine the replaceable sensor portion has been replaced with a new sensor portion;
determine, based on the one or more signals indicative of the concentration of the one or more analytes over the second period of time, a concentration of the one or more analytes over the second period of time;
determine, based on the patient-specific relationships, that the concentration does not satisfy a threshold similarity with the estimated concentration of the one or more analytes; and
calibrate the new sensor portion based on the EGM for the patient collected over the second period of time.

5. The system of claim 1, wherein the cardiac EGM comprises an electrocardiogram (ECG), and wherein the processing circuitry is configured to determine the one or more patient-specific relationships between the one or more signals and characteristics of the ECG.

6. The system of claim 5, wherein the characteristics comprise one or more of: an RT interval, a QT interval, T-wave amplitude, T-wave inversion, U-wave visibility, ST segment depression, R-wave amplitude, or QRS width.

7. The system of claim 1, wherein to determine the one or more patient-specific relationships between the one or more signals and the cardiac EGM, the processing circuitry is configured to:
train a machine learning model to determine the estimated concentration of the one or more analytes as output based on an input cardiac EGM segment using a set of training data that includes the one or more signals indicative of the concentration of the one or more analytes and the cardiac EGM, wherein each segment of the one or more signals includes a label indicating a corresponding segment of the cardiac EGM.

8. The system of claim 1, wherein each work electrode of the one or more work electrodes comprises a respective membrane disposed on a respective reagent substrate, and wherein the respective membrane is selectively permeable to the respective analyte.

9. The system of claim 1, wherein the first period of time comprises a week.

10. The system of claim 1, wherein the processing circuitry is further configured to determine, based on the patient-specific relationships and the one or more signals produced over a third period of time, an estimated heart rate variability for the patient.

11. A method comprising:
producing, by an electrochemical sensor comprising one or more work electrodes, one or more signals indicative of a concentration of one or more analytes in a patient;
sensing, by an implantable medical device (IMD), a cardiac electrogram (EGM) of the patient;
determining, by the processing circuitry, one or more patient-specific relationships between the one or more signals indicative of the concentration of the one or more analytes over a first period of time and the cardiac EGM for the patient collected over the first period of time; and
based on the patient-specific relationships, determining, by the processing circuitry, an estimated concentration of the one or more analytes based on the EGM for the patient collected over a second period of time.

12. The method of claim 11, wherein a biocompatible medical device comprises the electrochemical sensor and is configured for insertion through skin and into interstitial fluid of the patient.

13. The method of claim 11, further comprising:
from the electrochemical sensor, receiving, by the processing circuitry, a signal from each of a first work electrode comprising a first reagent substrate configured to react with potassium to produce a signal indicative of a concentration of potassium and a second work electrode comprising a second reagent substrate configured to react with glucose to produce a signal indicative of a concentration of glucose, wherein the first work electrode and the second work electrode are electrically coupled to a common counter electrode and a common reference electrode;
identifying, by the processing circuitry, one or more of a signal corresponding to the first work electrode and a signal corresponding to the second work electrode; and
based on the identified signal, determining, by the processing circuitry, one or more of a concentration of potassium and a concentration of glucose associated with the first work electrode or the second work electrode, respectively.

14. The method of claim 11, further comprising:
determining, by the processing circuitry, that a replaceable portion of the electrochemical sensor comprising one or more work electrodes has been replaced with a new sensor portion;
based on the one or more signals indicative of the concentration of the one or more analytes over the second period of time, determining, by the processing circuitry, a concentration of the one or more analytes over the second period of time;
based on the patient-specific relationships, determining, by the processing circuitry, that the concentration does not satisfy a threshold similarity with the estimated concentration of the one or more analytes; and
calibrating, by the processing circuitry, the new sensor portion based on the EGM for the patient collected over the second period of time.

15. The method of claim 11, wherein the cardiac EGM comprises an electrocardiogram (ECG).

16. The method of claim 15, wherein determining the one or more patient-specific relationships comprises:
determining, by the processing circuitry, relationships between the one or more signals and characteristics of the ECG, wherein the characteristics comprise one or more of: an RT interval, a QT interval, T-wave amplitude, T-wave inversion, U-wave visibility, ST segment depression, R-wave amplitude, or QRS width.

17. The method of claim 11, wherein determining the one or more patient-specific relationships between the one or more signals and the cardiac EGM comprises:
training, by the processing circuitry, a machine learning model to determine the estimated concentration of the one or more analytes as output based on an input cardiac EGM segment using a set of training data that includes the one or more signals indicative of the concentration of the one or more analytes and the cardiac EGM, wherein each segment of the one or more signals includes a label indicating a corresponding segment of the cardiac EGM.

18. The method of claim 11, wherein each work electrode of the one or more work electrodes comprises a respective membrane disposed on a respective reagent substrate, and wherein the respective membrane is selectively permeable to the respective analyte.

19. The method of claim 11, further comprising:
based on the patient-specific relationships and the one or more signals produced over a third period of time, determining, by the processing circuitry, an estimated heart rate variability for the patient.

20. A non-transitory computer-readable storage medium, comprising instructions that, when executed by processing circuitry, cause the processing circuitry to:
determine one or more patient-specific relationships between the one or more signals indicative of the concentration of the one or more analytes over a first period of time and the cardiac EGM for a patient collected over the first period of time; and
based on the patient-specific relationships, determine an estimated concentration of the one or more analytes based on the EGM for the patient collected over a second period of time.
